(19) United States
(12) Patent Application Publication
(10) Pub. No.: US 2009/0198145 A1
(43) Pub. Date: Aug. 6, 2009

(54) COMPOSITIONS, METHODS, AND SYSTEMS FOR RAPID INDUCTION AND MAINTENANCE OF CONTINUOUS REM SLEEP

(76) Inventor: Harrison CHOW, Saratoga, CA (US)

Correspondence Address: LEVINE BAGADE HAN LLP, 2400 GENG ROAD, SUITE 120, PALO ALTO, CA, 94303, US

(21) Appl. No.: 12/330,427

(22) Filed: Dec. 8, 2008

Related U.S. Application Data
(60) Provisional application No. 61/026,696, filed on Feb. 6, 2008.

(51) Int. Cl. A61B 5/0488; A61K 31/05; A61K 31/135; A61K 31/4164; A61K 31/661; A61P 25/00; A61K 38/02; A61K 31/137; A61K 31/167; A61K 31/5517; A61K 31/4468; A61K 31/573; A61K 31/4178; A61B 5/0476; A61B 5/11; A61M 31/00

(52) U.S. Cl. 600/544; 514/731; 514/647; 514/400; 514/130; 514/2; 514/646; 514/626; 514/220; 514/329; 514/180; 514/397; 600/546; 600/595; 604/500

(57) ABSTRACT

Compositions, kits, methods, and systems to induce, maintain, monitor, and interpret a continuous, un-fragmented REM sleep state in humans, generate dreams, recover sleep, create brain neuronal plasticity and activate the brain for cognition enhancement and mood stabilization are described. If potentially combined with other medications, a platform is provided to develop new research and therapies in many fields treating the human brain. The procedure reliably and quickly generates a Continuous REM Sleep cycle of pre-determined time or indefinite duration depending on therapeutic goals, allowing the patient to experience a qualitatively superior dream sleep in a shortened period of time compared to a natural sleep cycle. Profuse positive (pleasant) dreams are produced as well. REM sleep, dreams and sleep recovery can be reliably generated, and various sleep, psychological and neurological illnesses and disorders can be treated or prevented either solely by this method or in combination with additional agents.

### CROSS-REFERENCE TO RELATED APPLICATIONS

This application claims the benefit of priority to U.S. Prov. Pat. App. 61/026,696 filed Feb. 6, 2008, which is incorporated herein by reference in its entirety.

### BACKGROUND OF THE INVENTION

Sleep is postulated to be a state of natural rest and physiological recovery, and is seen in all mammals, and throughout the animal kingdom. Sleep in humans is characterized by a reduction in voluntary body movement, decreased reaction to external stimuli, and loss of consciousness. Sleep has been implicated in learning, memory formation, cellular restoration, brain development, mood regulation and stress relief in humans.

There are five internationally recognized stages of sleep, as characterized by a sleeping individual's electroencephalogram (EEG). Non-rapid eye movement sleep (NREM) ranges in sleep depth from Stage 1 (light sleep) through Stage 4 (deepest sleep). Stage 1 sleep is drowsiness, in which the EEG displays a lower voltage, more mixed frequencies, and a deterioration of alpha rhythm, relative to the EEG exhibited when an individual is awake. In Stage 2, background activity similar to that of Stage 1 is experienced, with slightly higher frequency “sleep spindles” and sporadic higher amplitude of slow wave complexes. Stage 3 and Stage 4 sleep, also known as slow wave sleep (SWS), display increasingly higher amplitude slow wave activity and are considered “deep sleep”. Typically, the vast majority of total sleep time is spent in NREM sleep. Rapid eye movement (REM) sleep cycles are often brief and fragmented compared to other sleep cycles with the majority of dreams occurring during REM sleep.

Dreams have historically been a source of keen interest and inspiration in many human cultures and are thought to occur primarily during REM sleep. Dreams are defined as an experience involving a sequence of images, sounds, ideas, emotions or other sensations occurring during sleep.

REM sleep is characterized by high frequency, low amplitude, desynchronized EEG waves, rapid ocular muscle movements and low muscle tone. The function of dreams remain unclear with many researchers postulating that dreams produced during REM sleep have a critical role in neural processes involving learning, memory consolidation, and mood regulation. REM sleep is also considered fundamental to providing sleep recovery and psychological rest. Neurologically, REM sleep originates from neurons in the brainstem that propagate through neurotransmitters to the neocortex. Molecular neurotransmitters such as norepinephrine, acetylcholine, dopamine, serotonin, and cortisol are all postulated to be critical mediators in endogenous REM sleep physiology.

The relationship and interaction between anesthesia-induced hypnosis and natural sleep neurobiological mechanisms have been a source of interest amongst anesthesia researchers. Natural sleep cycles have been induced and maintained using anesthesia techniques in rats. However, it is believed that continuous and un-fragmented REM sleep has not previously been selectively induced or maintained using anesthetics or other pharmacological agents.

Stanford University psychiatrist and sleep researcher, Dr. William Dement, postulated that sleep can be delayed for long periods of wakefulness but “sleep debt” eventually needs to be “repaid” in equivalent linear amounts of sleep time in order to achieve psychological sleep recovery. However recent evidence suggests that REM sleep-rich naps may quickly provide sleep recovery and psychological benefits of sleep in a much shorter time.

The mental and physical disruptions of poor sleep caused by sleep deprivation, stressors, or psychological disturbances are well documented, and often lead to negative health, personal, and financial consequences. Acute and chronic sleep disturbances have been strongly implicated as a causal factor in traffic and other operator-dependent accidents, jet-lag, and decreased employee productivity; they may also play a significant role in illnesses connected to psychological conditions, including but not limited to post-traumatic stress disorder (PTSD), depression, anxiety disorders, and chronic pain.

REM sleep disturbances have been documented as contributing to the psychological symptoms of sleepiness, memory loss, and mood instability associated with obstructive sleep apnea, restless leg syndrome and anxiety disorders such PTSD. Though the effects of REM sleep deprivation in humans are still unclear, prolonged REM sleep deprivation in animal populations has led to death.

The relationship of human cognition processes, learning and memory, to sleep currently is of great interest to researchers. Neurons in the brainstem, hippocampus, cortex, and neocortex are thought to be active in storing and consolidating memories or learning during sleep. REM sleep, in particular, is postulated to have a primary role in changes (plasticity) in the brain and the neuronal synaptic efficacy involved in storing new memories from awake experiences, consolidating, and re-consolidating old memories into stable memories, and supporting new learning.

Current pharmaceutical development for learning and memory therapy is mostly in investigative stages. Collectively, such drugs or “cognition enhancers”, to-date have had limited if any demonstrated effectiveness in rehabilitating degenerative memory and learning losses associated with Alzheimer's disease, senile dementia, Parkinson's, and stroke. New directions, which may be further advanced by this invention, involve altering the presence, concentration or efficacy of neurotransmitters (glutamate, serotonin, dopamine etc) at specific receptors (NMDA/AMPA etc) at neuronal synapses of neural networks of the brain affecting mood and cognition (memory and learning) processes.

Current pharmacological sleep recovery therapy is focused on using oral sedatives such as benzodiazepines (including diazepam (e.g., VALIUM®)) and cyclopyrrones (including zolpidem (e.g., AMBIEN®)) to initiate or promote sleep cycle induction. The use of oral sedatives, however, may be limited by rapid tolerance, abuse, and overdose potential. Furthermore, such oral sedatives often inhibit REM sleep and dreams, and are also associated with post-sleep “hangover” sedation. Thus, the effectiveness of these oral sedatives appear to be variable and limited, particularly for learning and memory enhancement, which are typically associated with REM.

### SUMMARY OF THE INVENTION

The present invention provides innovative compositions, processes, methods, kits, and systems for rapidly and predictably inducing and maintaining continuous rapid eye movement sleep, resulting in the reliable production of dreams and rapid sleep recovery to treat sleep-affected disorders as well as primary psychological and neurological disorders. In addition, the present invention may create a REM-specific “plastic” neuronal state in the brain and thereby activate and enhance the function of cognition (memory and learning) and mood stabilization processes and pathways in the brain.

In the present invention, REM sleep is selectively induced and continuously maintained using a specific pharmacological induction and maintenance process with little or no fragmentation into other NREM sleep stages (Stages 1-4). By use of this invention, patients report a full perception of sleep recovery in as little as 20 minutes, in contrast to typical 6-8 hours. This is the first instance, known to the inventor, where patients can experience REM sleep in a continuous and unfragmented state for either a predetermined period of time, or alternatively, for an indefinite duration.

Using specific pharmacologic agents and processes described herein, “continuous stimulation” of REM-producing neurons can be generated without interruption for an indefinite duration so long as the pharmacological and monitoring process is maintained (“Continuous REM Sleep”). Accordingly, Continuous REM Sleep may be produced in a patient to last for a specified period of time for a Continuous REM Sleep Cycle; alternatively, the Continue REM Sleep may be maintained for as long a period of time as necessary or desirable, e.g., indefinitely. In contrast, in natural sleep, REM sleep often does not occur, and if it does occur, REM is highly fragmented, and typically limited to 15-30 minutes, before transitioning to other stages of sleep.

Continuous REM Sleep is characterized by clinical signs consistent with natural “REM sleep,” including REM-specific high frequency, low amplitude, desynchronized electroencephalogram (EEG ) wave activity, rhythmic (saccadic) ocular muscle activity, phasic electromyelogram (EMG) activity, the psychological production of dreams, generalized muscle hypotonia, and rapid sleep recovery.

According to the present invention, Continuous REM Sleep can be reliably produced by the use of one or more pharmacologic agents (e.g., a gamma-aminobutyric acid type A receptor (GABA-A agonist) given in the specific invented process, in conjunction with other adjunct medications.

According to the present invention, Continuous REM Sleep can produce the psychological effects of sleep recovery and dreams chronologically faster than typical natural sleep cycles. Patients perceive a full night(s) or many hours of restful sleep time and dreams (and often superior quality of sleep than experienced with natural sleep) in as little as 20 minutes of Continuous REM Sleep.

The production of Continuous REM Sleep can also reset the circadian diurnal (day) and nocturnal (night) sleep clock cycle. After Continuous REM Sleep, patients report an immediate enhanced promotion of natural sleep tied to environmental cues (day/night cycles), and sleep better in subsequent nights.

According to the present invention, Continuous REM Sleep may also promote learning and memory (cognition enhancement) in patients. Continuous REM Sleep may promote learning and memory by facilitating the storage of new information from the awake state into new memories, consolidating these memories into a permanent state (long term memory), reconsolidating stored memories to stabilize long term memories, and consolidating relationships between these memories into learning. Continuous REM Sleep may be used to treat psychological and neurological illnesses and disorders that are affected by memory and learning abnormalities.

According to the present invention, the methodology of Continuous REM Sleep may create synaptic plasticity in brain neurons that promote learning and memory processes. Molecules or agents that promote memory acquisition, consolidation, reconsolidation and learning, collectively known as cognition enhancers, may be functionally activated or enhanced during Continuous REM Sleep.

According to the present invention, the methodology of Continuous REM Sleep may create synaptic plasticity in brain neurons that affect emotions and mood regulation. Molecules that promote emotional and mood stability may be activated or enhanced during Continuous REM Sleep.

According to the present invention, the methodology of Continuous REM Sleep by generating neuronal synaptic plasticity, may provide a therapeutic and experimental platform for molecules, drugs, and processes designed to enhance or stabilize human cognition (cognition enhancers).

According to the present invention, the methodology of Continuous REM Sleep by generating neuronal synaptic plasticity, may provide a therapeutic and experimental platform for molecules, drugs, and processes designed to enhance or stabilize human emotions and mood (mood stabilizers).

As used herein, “Continuous REM Sleep” is defined as the exposure to a hypnotic agent for an unlimited and undefined time using a specific process (described herein) to generate the production of a continuous and un-fragmented REM sleep state in a human subject.

As used herein, a “Continuous REM Sleep Cycle” is defined as the time of exposure to a hypnotic agent using the processes described herein, preferably 5-240 minutes, more preferably 10-120 minutes, and most preferably 20-60 minutes, or an indefinite duration.

According to the present invention, the hypnotic pharmacological agent may be any sedative (promoting or aiding unconsciousness) and hypnotic (producing unconsciousness) agent, such as benzodiazepines, cyclopyrrones, neurosteroids, barbiturates, alcohol, narcotics, and anesthetics (e.g., etomidate, propofol, fospropofol, and ketamine). Preferably, the hypnotic or sedative agent is a GABA-A agonist which causes sedation and hypnosis effects primarily through selective GABA-A agonist action in the brain. In one embodiment, the hypnotic or sedative agent is propofol, or its prodrug, metabolite, analog, and derivative.

According to the present invention, Continuous REM Sleep can also be produced by using other anesthetic hypnotic agents, including volatile gases, nitrous oxide, barbiturates, analogs, derivatives and metabolites thereof given in a similar process.

According to the present invention, the hypnotic or sedative agent may also be used in conjunction with other medications including anti-emetics (e.g., steroids, and 5-HT blockers), antidepressants, anti-psychotics, narcotics, benzodiazepines, local anesthetics and ketamine.

According to the present invention, the hypnotic or sedative agent may also be used in conjunction with other medications that may affect the production or content of REM sleep and dreams including drugs that are analogs or substitutes that promote, decrease or alter neuronal synaptic concentrations and receptor sensitivity of neurotransmitters in REM sleep neural pathways, including narcotics, benzodiazepines, neurosteroids, serotonin, dopamine, norepinephrine, acetylcholine and other potential neuromodulators.

According to the present invention, Continuous REM Sleep production, may initiate, promote and maintain neuronal synaptic plasticity in neural pathways that affect cognition (memory and learning) and mood regulation.

According to the present invention, the production of Continuous REM Sleep, by creating neuronal synaptic plasticity, may augment or initiate the synaptic and therapeutic efficacy of cognition enhancement and mood stabilization drugs and therapy.

According to the present invention, the production of Continuous REM Sleep, by creating neuronal synaptic plasticity, may augment or initiate cognition enhancement therapy when given in specific REM time window for memory consolidation, learning and mood stabilization benefits. Such efficacy time windows may involve simultaneous administration of cognition enhancement and/or mood stabilization agents during and/or prior to, and/or subsequent to Continuous REM Sleep generation.

Continuous REM Sleep production may be monitored for hypnotic depth (level of consciousness or LOC) and by observed specific clinical signs of EEG REM-characteristic wave forms, phasic electromyelogram (EMG) activity by a brain function monitor such as a commercially-available EEG brain function monitor used in the field of anesthesiology. Ocular muscle (eyeball) movement consistent with REM sleep can be observed independently by visual confirmation.

Continuous REM Sleep can be safely produced by the use of delivered oxygen, intravenous access, the use of standard anesthetic monitoring (pulse oximetry, capnometry, EKG, non-invasive blood pressure) with the drug delivery and patient management and monitoring by a qualified health care provider, preferably an anesthesiologist.

According to the present invention, production of Continuous REM Sleep can be used to clinically recover the psychological effects of sleep deprivation and to treat sleep-related disorders and illnesses associated with poor quality sleep recovery.

According to the present invention, production of Continuous REM Sleep can be used to clinically treat the psychological effects associated with altered or poor quality sleep restfulness associated with psychological disorders and illnesses, or to clinically treat the psychological effects associated with deficient or poor quality sleep associated with mood disorders and illnesses.

According to the present invention, production of Continuous REM Sleep can be used to clinically treat the psychological effects associated with the lack of dreams or negative dreams (nightmares, night terrors) associated with psychological disorders and illnesses.

According to the present invention, production of Continuous REM Sleep can be used to clinically treat and reduce the symptoms of psychological disorders and illness, through the rich and profuse production of dreams.

According to the present invention, production of Continuous REM Sleep can be used to provide a clinical means of producing dreams for the purposes of therapeutically treating human stress and anxiety disorders.

According to the present invention, production of Continuous REM Sleep can be used to clinically assist the synchronization of natural sleep cycles and recovery of psychological symptoms related to circadian rhythm and cycle disturbances.

In one aspect of the invention, a method is provided for inducing and maintaining Continuous REM Sleep in a subject. In one embodiment, the method comprises: administering to the subject a pharmaceutically effective amount of a hypnotic or sedative agent such that GABA-mediated inhibition of brain neuronal activity results in REM neuronal activity stimulation for a period of time of 5-240 minutes, or for an indefinite duration.

In another embodiment, the method comprises: administering to the subject a pharmaceutically effective amount of a first hypnotic agent such that the level of consciousness (LOC) of the subject is reduced to lower than 70%, optionally lower than 60%, 50%, 40%, 30%, or 25%, as compared to the LOC measured when the subject is awake, thereby inducing a Continuous REM Sleep in the subject. Preferably, a hypnotic or sedative agent is a GABA-A agonist, such as propofol, which is administered to the subject via bolus injection.

The method may further comprise the step of administering a second hypnotic agent to the subject continuously for a period of time at a dose such that the subject remains in hypnosis and the LOC of the subject is maintained during this period of time at about 50-90%, 60-85%, 65-85%, or 70-80% as compared to the LOC measured when the subject is awake, thereby maintaining Continuous REM Sleep in the subject.

According to the method, the first and second hypnotic agent may be the same or different. For example, the first and second hypnotic agent can both be propofol. Alternatively, the first hypnotic agent may be propofol, and the second agent may be another, different GABA-A agonist or other brain active agent(s).

In yet another aspect of the invention, a method is provided for inducing and maintaining a Continuous REM Sleep Cycle in a subject, comprising: administering to the subject a pharmaceutically effective amount of a first hypnotic agent such that the level of consciousness (LOC) of the subject is reduced to lower than 70%, optionally lower than 60%, 50%, 40%, 30%, or 25%, as compared to the LOC measured when the subject is awake. Preferably, a hypnotic or sedative agent is a GABA-A agonist such as propofol which is administered to the subject via bolus injection.

The method may further comprise the step of administering a second hypnotic agent to the subject continuously for a period of time at a dose such that the subject remains in hypnosis and the LOC of the subject is maintained during this period of time at about 50-90%, 60-85%, 65-85%, or 70-80% as compared to the LOC measured when the subject is awake. Termination of the administration hypnotic agent (after a predetermined time) to the subject will result in the awakening of the subject. The period of time of a Continuous REM Sleep cycle is preferably 5-240 minutes, 10-180 minutes or 20-60 minutes, or an indefinite duration.

According to the method, the first and second hypnotic agent may be the same or different. For example, the first and second hypnotic agent can both be propofol. Alternatively, the first hypnotic agent may be propofol, and the second agent may be another, different GABA-A agonist or other brain active agent(s).

In yet another aspect of the invention, a novel cerebral electroencephalogram (EEG) monitoring system (“REM Machine”) is provided for the monitoring the induction, maintenance, and interpretation of Continuous REM Sleep cycle in a subject and document the production of dreams and REM activity as necessary for therapeutic goals of sleep recovery, memory and learning and mood stabilization. Such a system designed specifically for Continuous REM Sleep monitoring will allow the clinical practitioner to adjust pharmacological administration to specific clinical goals of initiating and maintaining Continuous REM Sleep for pre-determined time. Such a system could be designed to interpret REM EEG waveforms using computerized spectral analysis algorithms, such as the fast Fourier transform (FFT) test, to identify and analyze specific dream and cognition activity, quantify REM EEG wave intensity (cycles per second) and to document post-procedure dreams, cognition (memory and learning) and mood changes. Such a system along with REM EEG analysis, could also measure other forms of REM or sleep state activity (if at all present), including slow wave sleep (SWS) and time ratios and sequences between other sleep stages and REM sleep.

Such a system or “REM Machine” may be a monitor configured and developed using specific electronic algorithms to assess and weight variables used in monitoring Continuous REM Sleep production. These variables include (but are not limited to) level of consciousness (LOC) score, electromyelogram (EMG) activity score, ocular muscle (OM) activity score (eyelid sensor), and the presence of REM-specific low amplitude/high frequency/desynchronized EEG waveform and rhythmic (saccadic) eyelid activity. An eyelid sensor, specifically placed on a patient's eyelid (FIG. 6), may be added over presently available brain function monitors to measure OM activity directly. The eyelid lead would measure the saccadic displacement of the eyelid caused by the underlying ocular muscle (eyeball) movement seen in REM sleep and measured in cycles per second (cps). This system may provide a total score for the presence of these variables (LOC+EMG+OM+EEG) at preset levels to produce a single numerical score to alert the practitioner of the successful initiation and presence of Continuous REM Sleep.

REM-specific dream content and cognition processes would be identified by this “REM Machine”. Monitors can be calibrated to recognize specific REM EEG patterns using spectral frequency analysis algorithms, electronically correlate these EEG patterns or “REM Profiles” with REM EEG indices of dream content and cognition processes. Such a REM Profile may be comprised at least of REM frequency changes obtained from measured REM EEG patterns. Additionally, a REM Profile can also include other data such as EMG activity scores, OM activity scores, as well as LOC scores. The monitor can be configured to track a specific type of dream content or cognition process, intensity of REM activity (cycles per second) and time spent in REM and other sleep stages such as SWS (if present at all). The monitor may also allow for input of, e.g., specific patient information, medical history, treatment plan, specific drugs used (anesthetics, cognition enhancers, mood stabilizers etc.) and documented efficacy of sleep recovery, mood and cognition changes.

The method may further include providing the hypnotic agent(s), related adjunct medications, cognition enhancement or mood stabilization agents, systems, kits, and instructions, either directly or as a consultant, for use of the agent to a physician or health care provider for administration to a subject (patient) in need of treatment, prevention or alleviation of sleep-related or sleep-affected diseases or disorders. Such instructions for use of the hypnotic agent(s), adjunct medications, cognition enhancement or mood stabilizing agents, systems, kits can include the methods and procedures described herein. The method may optionally include billing the patient or the patient's insurance provider. The method may also include providing kits disclosed herein to a physician or health care provider.

The compositions, methods, kits and systems can be used for treating, preventing or alleviating symptoms of a wide variety of psychological and sleep-affected illnesses or disorders, such as insomnia; psychological conditions such as major depression, hypomania, cyclothymia, anxiety, bipolar disorder, hyperactivity, attention deficit disorder, chronic fatigue syndrome, premenstrual syndrome (PMS), premenstrual dysphoric disorder (PMDD), and agoraphobia; stress-related disorders as chronic fatigue syndrome (CFS), fibromyalgia (FMS), Gulf War Syndrome; anxiety disorders such as post-traumatic stress disorder (PTSD); and circadian rhythm abnormalities such as jet lag, shift work sleep disorder and seasonal affective disorder (SAD); and general dream therapy for mood stabilization.

The compositions, methods, kits and systems can also be used for treating, preventing or alleviating symptoms of psychological disorders, such as those Class 5 mental disorders according to “International Classification of Diseases” (ICD), 9th Revision, Clinical Modification, Seventh Edition, 2007 or ICD-9-CM 2007.

The compositions, methods, kits and systems can also be used for treating, preventing or alleviating symptoms of neurological disorders, such as those Class 6 neurological disorders according to “International Classification of Diseases” (ICD), 9th Revision, Clinical Modification, Seventh Edition, 2007 or ICD-9-CM 2007.

The compositions, methods, kits and systems can also be used in the for-profit or not-for-profit research of sleep, sleep-related disorders, stress-related disorders, psychological disorders, or neurological disorders.

The compositions, methods, kits and systems can also be used for recreational purposes, such as for general stress relief for healthy individuals and for improving quality of life and mental health.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart schematically illustrating the proposed neuronal mechanisms of action for pharmacological induction of Continuous REM Sleep according to the present invention.

FIG. 2 is a flow chart schematically illustrating an embodiment of the inventive methodology for the induction, maintenance and termination of a Continuous REM Sleep cycle.

FIG. 3 is a graphical illustration of the dosage of propofol administered to a person over a 60 minute Continuous REM Sleep Cycle.

FIG. 4 is a graphical illustration of an approximate percentage of level of consciousness (LOC) of the person over a 60 minute Continuous REM Sleep cycle as in FIG. 3.

FIG. 5 is a 30 second EEG waveform trace (high frequency, low amplitude, desynchronized wave pattern plus ocular muscle movement artifact) of a patient in a Continuous REM Sleep state using the commercially available brain monitor (also known as a “BIS” bispectral index Vista monitor) by Aspect Medical Systems (Norwood, Mass.).

FIG. 6 is a drawing of a brain function monitor enhancement incorporating a R (REM) sensor placed on the lateral eyelid border (lateral eyelid margin) to capture saccadic eyelid displacement caused by underlying ocular muscle activity and eyeball movement data.

FIG. 7 is a flow chart schematically illustrating the exemplary use of cognition enhancement and/or mood stabilization therapy during a Continuous REM Sleep cycle. Cognition enhancement and/or mood stabilization therapy may involve the administration of cognitive enhancing or mood stabilizing agents prior to, during, or after a Continuous REM Sleep cycle.

### DETAILED DESCRIPTION OF THE INVENTION

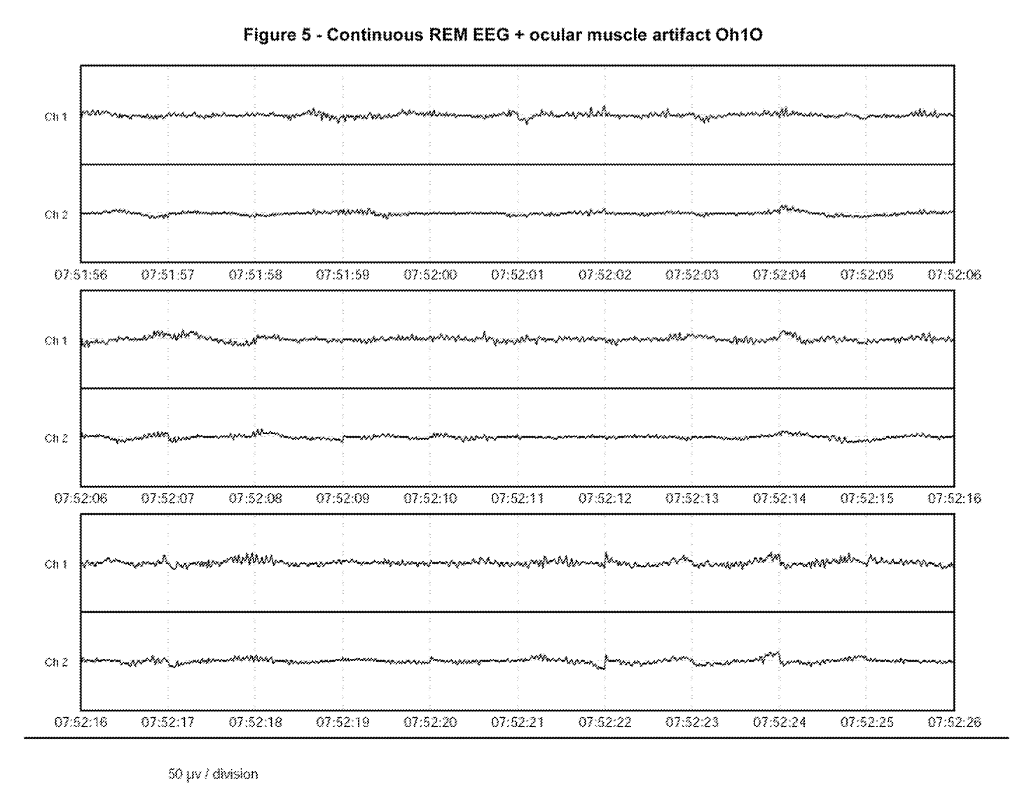

The present invention provides an innovative pharmacological process using hypnotic agents to induce and maintain dreams, as well as Continuous REM Sleep. This invention describes the discoveries that control, monitor, and administer the hypnotic or sedative agent such as a GABA-A agonist (e.g., propofol) and adjunctive medications at designed dosages, timing, and modes of administration to produce dreams and an unique sleep state of un-fragmented Continuous REM Sleep. In the inventor's clinical studies, over 80 patients achieved Continuous REM Sleep through application of the inventions as demonstrated in Example 1 and described herein. Clinical and cerebral monitoring during the administration of pharmacological agents using the specific invented process are consistent with natural REM sleep, including clinically observed characteristic electroencephalogram (EEG) REM-specific waveforms FIG. 5), saccadic ocular muscle activity (FIG. 5), phasic electromyelogram (EMG) activity and concomitant generalized body hypotonia (muscle relaxation). Patients, upon waking, report dreams and rapid sleep recovery consistent with the accepted clinical description of natural REM sleep (Aserinsky E, Kleitman N. 1953. Regularly Occurring Periods of Eye Motility, and Concomitant Phenomena during Sleep. Science, Vol 118; 273-274).

However, the clinical profile of Continuous REM Sleep production is distinct from a natural sleep state (including natural REM sleep) in the reliability of production, rapid chronological generation, the un-fragmented continuous maintenance, and patient-reported enhanced quality of dreams and sleep recovery. The present invention describes the use of a specific hypnotic agent, the GABA-A agonist propofol, to suppress specific cerebral neurons (more specifically, suppress REM-OFF neurons). The infusion of propofol and the addition of adjunct medications result in “continuous stimulation” of REM neurons in the sleeping brain. Such continuous REM neuronal activity and REM production clinically produces a patient perception of rapid and enhanced sleep recovery and dreams in relatively short periods of time.

The overlapping relationship between anesthesia and natural sleep pathways has been a source of interest to researchers. Propofol given intravenously was demonstrated to replicate a natural sleep cycle, along with alternating NREM and REM sleep stages, over a 6 hour period in rats (Tung et al. Recovery from sleep deprivation occurs during propofol anesthesia; 2004, Anesthesiology, Vol 100: 1419-26). Aspects of this invention include the selective and predictable induction and continuous maintenance of REM sleep using a combination of pharmacological agents, and the production of such REM sleep in a continuous form with minimal or no fragmentation into other NREM sleep stages (Stages 1-4). Patients who have benefited from Continuous REM Sleep report full sleep recovery in as little as 20 minutes as opposed to a typical 6-8 hour natural sleep cycle.

The inventor is able to induce and maintain a dream state among human subjects. While dreaming following anesthesia administration for surgical procedures has been previously observed, previous studies have found no relationship between depth of anesthesia (level of consciousness or LOC) and the relatively low incidence of dreaming (22%) found after anesthesia administration in post-operative patients (Leslie et al. Dreaming during anesthesia and anesthetic depth in elective surgery patients; 2007, Anesthesiology, Vol 106:33-42). In contrast, according to this invention and the inventor's clinical observations of a prospective group of more than 80 surgical patients, dreaming is induced with high incidence (>75%) using this invention; dreaming is also induced with relatively high levels of consciousness (LOC>60, preferably LOC>70), with clinical signs of REM sleep present (REM EEG waveform, saccadic ocular muscle movement, phasic EMG activity, and generalized muscle hypotonia). Moreover, most of these patients, who recalled dreams during Continuous REM Sleep, often relayed “positive” or pleasant dreams. Even among the patients who did not report dreams, they may have still have experienced dreams during the invented process, but were unable to recall or were amnestic. All of these patients, most who had slept poorly the night before surgery, reported a psychological perception of full and enhanced sleep recovery.

The Bispectral Index (BIS) EEG monitor, primarily developed for use to measure level of consciousness under anesthesia, has also been used to measure depth of sleep and document sleep stages during natural sleep (Sleigh, J. et al. The Bispectral Index: A measure of depth of sleep?; 1999, Anesthesia and Analgesia, Vol 88: 659-667.). Natural REM sleep, reflected in the BIS monitor EEG waveforms, was noted at BIS levels of consciousness (LOC) of 75-92 during natural sleep. In this invention, REM sleep and clinical activity occurred at relatively high levels of consciousness (LOC>60, preferably LOC>70), consistent with prior BIS research on natural sleep.

Natural sleep follows a typical pattern of periodicity comprising 80-110 minute cycles with a sequence of light sleep (stages 1 and 2) leading to deep sleep (stages 3 and 4) and to REM sleep. Natural sleep, normally over a 6 to 8 hour period of continuous mixed-stage sleep, comprises 4 to 6 such short cycles (starting in a linear manner at stage 1 and leading to REM.). Natural sleep and REM is, thus, normally “fragmented” by different sleep stages, and even by short awakenings or “arousals” (Borbely, A. and Achermann, P., 1999, Sleep homeostasis and models of sleep regulation, J. Biol. Rhythms, Vol 14, pp. 557-568). Natural REM sleep is almost uniformly limited by 15-30 minute REM “sleep epochs” before transitioning to a NREM sleep state.

In some sleep disorders, sleep apnea and restless leg syndrome, REM sleep is severely curtailed or even absent, resulting in severe daytime sleepiness and poor sleep recovery (Penzel, T. et al., 2005, Analysis of sleep fragmentation and sleep structure in patients with sleep apnea and normal volunteers; Proceedings of the 2005 IEEE, pp 2591-2594). REM sleep can be critical to sleep recovery and the overall benefit of sleep; for example, REM-rich naps of 60 minutes have the same benefit on sleep recovery and learning as a 6-8 hour mixed-stage sleep period (Mednick et al., 2003, Sleep-dependent learning: a nap is as good as a night, Nature Neuroscience, Vol 6, Number 7, pp 697-698; Mednick et al., 2002. The restorative effect of naps on perceptual deterioration, Nature Neuroscience, Vol 5, Number 7, pp 677-681.). These results suggest that a Continuous REM Sleep cycle of 60 minutes or even less may have the same or possibly greater benefits on sleep recovery, cognition enhancement and/or mood stabilization than that provided by a natural sleep period of 6 to 8 hours.

As a result of natural and disorder-related fragmentation of sleep cycles, achieving REM sleep and dreams is brief, often difficult, or entirely elusive to many sleep patients having only limited time or a fraction of their total sleep cycle in a natural REM sleep state (Empson J., Sleep and Dreaming, Third Edition, 2002, pp 27-29). The controlled and rapid process of dreams and sleep recovery described in this invention contrasts greatly to the variability of dreams and sleep recovery experienced during natural sleep or other types of sleep production.

Continuous REM Sleep differs from natural and other pharmacologically-assisted sleep by generating a perception of superior quality of sleep and dreams in a short duration of time. In the inventor's clinical studies, patients often reported “that was a great night sleep”, “that was the best sleep I've ever had” and/or “those were the best dreams I've ever had”. Thus, the inventor believes that Continuous REM Sleep produces a qualitatively superior perception of sleep recovery and dreams than natural or other forms of pharmacologically-assisted sleep.

In an adult, natural sleep and a natural sleep cycle normally requires 6 to 8 hours (Browman, C. P. et al. 1977. “Reported sleep and drug use of workers: a preliminary report”, Sleep Research, Vol 6, 111). Typically, sleeping patients are aware of the actual linear time spent in sleep, and a time reference to the amount of sleep recovery in real time. However, Continuous REM Sleep production seems to differ from natural and other pharmacologically-assisted sleep by providing a chronologically rapid perception of sleep recovery. For example, despite the fact that a patient's Continuous REM Sleep cycle was as short as 20 minutes, patients often report sleep recovery consistent with a full natural sleep cycle of 6 to 8 hours.

Natural sleep is guided by endogenous diurnal/nocturnal regulation known as circadian rhythms or circadian sleep cycles (Halberg, F. 1969. Chronobiology. Annual Review of Physiology, Vol. 31, 675-725). Circadian sleep cycles maintain an internal clock to regulate natural sleep and are entrained by internal and external stimuli (day/night cues, light, anxiety, fatigue, perception of time, sleep deprivation) to maintain the optimal sleep cycle for function and awareness (Borbely, A. A. and Tobler, I. 1989. Endogenous sleep-promoting substances and sleep regulation. Physiological Reviews, Vol. 69, 605-670). The distortion of circadian clock cycle is associated with the negative psychological effects characterized by sleep deprivation and “jet lag”.

Continuous REM Sleep differs from natural and other pharmacologically-assisted sleep by not relying upon endogenous circadian cycles, and by altering the circadian clock cycle itself. In the inventor's clinical research, many patients achieving Continuous REM Sleep report enhanced ease of natural sleep initiation and maintenance of natural sleep the same night following daytime Continuous REM Sleep exposure, and with this effect occurring two to three nights subsequently. Thus, the inventor believes that Continuous REM Sleep generation appears to “reset” the circadian clock cycle by removing endogenous cues such as stress and natural sleep cycle time perception, thereby, allowing the circadian clock to more easily use exogenous cues (day/night, light perception) to initiate and maintain natural sleep.

According to the present invention, generation of Continuous REM Sleep may involve the controlled use of a hypnotic or sedative agent in combination with other adjunct medications, followed by rapid recovery of the patient to a waking state. Hypnotic agents, including but not limited to, propofol, barbiturates, narcotics, benzodiazepines, nonbenzodiazepine sedatives, psycholeptics, nitrous oxide and volatile anesthetic gases, are thought to render unconsciousness as GABA-A agonists, stimulating GABA-mediated inhibition of brain neuronal activity similar to sleep pathways (Nelson, L. et al., The sedative component of anesthesia is mediated by GABA-A receptors in an endogenous sleep pathway. 2002, Nature Neuroscience, Vol 5, number 10, 979-984). Details of GABA-A agonists (including propofol) and their roles played in sleep and sleep disorders are described in “Sleep and Sleep Disorders: A Neuropharmacological Approach, pp 3-7, 36-51, 135-145, edited by Lader, Carninali and Pandi-Perumal., 2006, Landes Bioscience and Springer Science, which is herein incorporated by reference. Pharmacological hypnosis (patient unconsciousness) though varied in dosage and clinical depth, is maintained throughout a Continuous REM Sleep cycle described in the present invention.

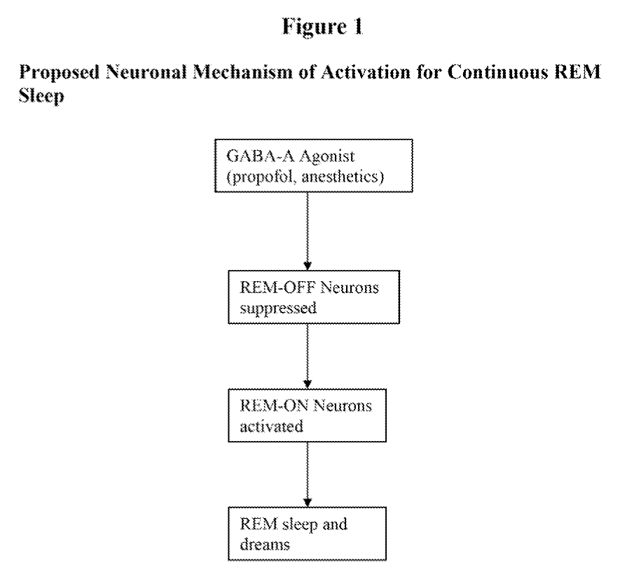

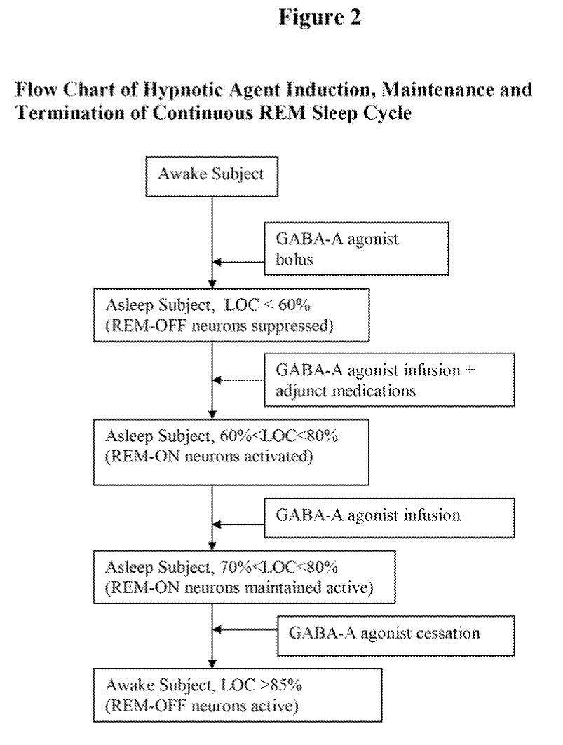

While not wishing to be bound to a particular theory or mechanism of action, the inventor believes that pharmacological generation of a Continuous REM Sleep cycle may include the following four steps: (i) hypnosis and suppression of REM-OFF brain neuronal activity; (ii) hypnosis and activation of REM-ON brain neuronal activity; (iii) hypnosis and maintenance of REM-ON brain neuronal activity; (iv) cessation of hypnosis and rapid recovery of brain neuronal wakefulness. The proposed neuronal mechanism of Continuous REM Sleep induction is outlined in a flow chart in FIG. 1, and an exemplary procedure for inducing, maintaining and terminating a Continuous REM Sleep Cycle by following steps (i)-(iv) is schematically illustrated in a flow chart in FIG. 2.

As illustrated in FIG. 2, in a first step (suppression of REM-OFF neurons) of a Continuous REM Sleep Cycle, the subject is administered a hypnotic agent. This agent rapidly induces deep hypnosis (loss of consciousness). At controlled bolus (large) dosages the hypnotic agent in addition to rendering deep unconsciousness (as measured by a brain function monitor) suppresses REM-OFF brain neurons at high blood concentration levels of the hypnotic agent.

In a second step (activation of REM-ON neurons) of a Continuous REM Sleep Cycle, the subject is administered the same hypnotic agent at a constant rate (infusion) at a dosage of approximately one-tenth the initial bolus dose. Related brain active agents or adjunct medications (5-HT uptake inhibitors, steroids and other potential brain agents, as well as any of the drug classes such as cognition enhancers and mood stabilizers that may affect the concentrations of these neurotransmitters or neuronal synaptic efficacy and as listed herein below) are administered at this time. At a starting constant infusion, the initial bolus hypnotic sedative agent given in step one redistributes and lowers in blood concentration in the brain, followed by the constant infusion of the second step. The inventor believes as REM-ON neurons are activated, continued suppression of REM-OFF neurons occurs by continued administration of hypnotic agents and REM-ON neuronal feedback inhibition of REM-OFF neurons.

In a third step (maintenance of REM-ON neurons) of a Continuous REM Sleep Cycle, the subject is administered the same hypnotic agent at a constant rate (infusion) to maintain light hypnosis. The infusion rate of the hypnotic agent is titrated in dosage and then maintained at constant rate based on the subject's level of consciousness.(brain function monitor and clinical signs) and characteristic REM EEG waveform while maintaining light hypnosis (measured by brain function monitor). The inventor believes as REM-ON neurons remain active at this step with minimal REM-OFF neuron activity, that physiologically maximal REM neuronal activity occurs, producing profuse and profound sleep recovery and dreams. Hypnotic agent dosage should be as light as possible to allow maximal positive dream recall. Continuous REM Sleep can be maintained indefinitely so long as this step is continued.

In a fourth step (cessation of hypnosis and recovery) of a Continuous REM Sleep Cycle, the hypnotic agent is discontinued and rapid consciousness, dream termination, and psychological wakeup (observed from clinical signs and brain function monitor) occurs as the blood concentration of hypnotic agent decreases. REM-OFF neurons are active and normal wakeful mechanisms are activated in maintaining awareness and alertness.

Current sleep and pharmacological research suggest that natural sleep, pharmacological sedation and hypnotic anesthetic actions are mediated by inhibitory neurotransmitter gamma-aminobutyric acid (GABA). Sleep and Sleep Disorder: A Neuropharmacological Approach, pp 3-7, 36-51, 135-145, edited by Lader, Carninali and Pandi-Perumal., 2006, Landes Bioscience and Springer Science. Hypnotic agents and anesthetics are thought to act specifically as an agonist at the GABA-A subunit of the GABA receptor complex. Important regions of the mammalian brain involved in sleep regulation include the hypothalamus (circadian cycles) and the midbrain reticular formation (MRF) of the brainstem (wakefulness and dreams). Both the hypothalamus and brainstem MRF, considered “primitive” brains in mammalian evolution, contain GABA receptors that regulate REM sleep as REM-OFF neurons. These REM-OFF neurons actively inhibit REM-ON neurons (MRF brainstem) during wakefulness and during most of sleep. (Mallick, B. N., Kaur, S. et al., Role of GABA in acetylcholine induced locus coeruleus mediated increase in REM sleep, Sleep Research, 1993, Vol. 22. 541.; Vanni-Mercier, G. Sakai, K. Jouvet, M. Waking state specific neurons in the caudal hypothalamus of the cat. C R Academy of Sciences 1984, Vol 298, 195-220.).

The inventor believes that Continuous REM Sleep production is a function of GABA receptor-mediated suppression of REM-OFF neurons that is mechanistically distinct and unique from natural REM sleep. During natural sleep, REM sleep is present for only a small fraction of total sleep time. During wakefulness and during most of natural sleep REM-OFF neurons inhibit REM-ON neurons (Hobson, J. A. and McCarley, R., “The brain as a dream state generator: an activation-synthesis hypothesis of the dream process”, American Journal of Psychiatry, 1997, Vol. 134, 1335-1348). In natural sleep, the brain, in a relaxed state, releases GABA, thus causing suppression of REM-OFF neurons and activation of REM-ON neurons in the brainstem, resulting in REM sleep. Return of REM-OFF activity suppresses REM-ON neurons and returns the brain to NREM sleep during natural sleep. During generation of Continuous REM Sleep, a GABA-A agonist hypnotic agent such as propofol suppresses REM-OFF neurons resulting in REM-ON neuronal activity and REM dream sleep (FIG. 1). Continued exposure to a hypnotic agent such as propofol as an infusion continues REM-OFF suppression and uninterrupted REM-ON neuronal activity under the proposed invention. The inventor believes that both the initial induction and maintenance of REM-OFF suppression by the use of a hypnotic agent causes greater and more sustained REM-ON neuronal activity than can be produced by natural sleep or other pharmacological means available prior to this invention. Clinically, after receiving a Continuous REM Sleep Cycle, the subjects reliably report substantially enhanced quality and length of time of sleep and dreams.

The induction and continued suppression of REM-OFF neurons by hypnotic agents causes considerable REM-ON neuronal activity. REM-ON neuronal activity creates REM dream sleep and while also simultaneously inhibiting REM-OFF neurons (FIG. 1). The inventor believes that during hypnotic agent induced REM-ON neuronal activity, REM-ON neurons are either sensitive to stimulation from other neurotransmitters either directly (adjunct medications) or indirectly through other regulatory neurons.

Adjunct agents used in this invention likely play an important role in stimulating or controlling REM-ON activity and Continuous REM Sleep. Such agents may include serotonin uptake inhibitors (ondansetron), steroids (dexamethasone), vasopressors (ephedrine), local anesthetics (lidocaine), narcotics (fentanyl), and benzodiazepines (midazolam). Serotonin, neurosteroids (cortisol) and norepinephrine, acetylcholine, dopamine, adenosine, glycine and glutamate are known neurotransmitters involved in REM sleep production and modulation (Payne, J and Nadel, L. Sleep, dreams, and memory consolidation: The role of the stress hormone cortisol, Learning and Memory, 2004 Vol 11: 671-678; Hobson, J et al., The neuropsychology of REM sleep dreaming. NeuroReport. 1998, Vol 9: R1-R14.). Other agents, in particular anti-depressants and anti-psychotic medications, may also affect REM sleep generation as well. The inventor believes that through use of the invention, which suppresses REM-OFF neurons concomitant with inducing REM-ON neuronal activity and stimulation, the initiation, length, density, and type of Continuous REM Sleep (and dreams) can be controlled, e.g., for therapeutic purposes.

The inventor also believes, that by practicing the present invention, disorders and illnesses affected by sleep disorders, circadian rhythm, cognition, and mood abnormalities may be treated. It is postulated that REM sleep and dreams in mammals and humans may play an important role in cognitive function, stress release, mood regulation, and memory formation. (Smith, C. “Sleep States and memory processes”, Behavioral Brain Research, 1995, Vol. 69, 137-145; Plihal, W. and Born, J. Effects of early and late nocturnal sleep on priming and spatial memory, Psychophysiology. 1999, Vol 36. 571-582; Greenberg, R. Dreams and REM sleep: An integrative approach. Sleep and Dreams and Memory 1981, 125-133, New York: Spectrum).

Recent cognition research has focused on the primary role of sleep on memory and learning in the human brain. Sleep is postulated to play an active role in processing awake memories into stable long-term memory in a neurological process known as “memory consolidation” (McGaugh, J., 2000, Memory—a century of consolidation, Science, Vol. 287, 248-251). Both REM and slow wave sleep (sleep stages 3 and 4) are thought to be critical towards creating long-term memory through memory consolidation (Walker, M. and Stickgold, R., 2004, Sleep-Dependent Learning and Memory Consolidation, Neuron, Vol. 44. pp 121-133). The presence of REM sleep during natural sleep has been proposed to be critical to the formation of both declarative (factually-based) and non-declarative (relationship or associative-based) memory (Smith, C., 2001, Sleep States and memory processes in humans: procedural versus declarative memory systems, Sleep Med. Rev., Vol 5, 491-506).

In particular, the role of REM sleep in forming weak associations and creative processing of stored memories in the brain have led researchers to propose that critical learning occurs during REM sleep (Walker et al., 2002, Cognitive flexibility across the sleep-wake cycle: REM-sleep enhancement of anagram problem solving, Cogn. Brain Rev., Vol. 14, 317-324; Stickgold, R. et al, 1999, Sleep-induced changes in associative memory, J. Cogn. Neurosci., Vol. 11, 182-193). The role of sleep, as a whole, in forming memory associations for “learning” have led researchers to propose that sleep may be the source of human “insight” (Wagner, U et al., 2004, Sleep inspires insight, Nature, Vol 427, 352-355). The present invention of Continuous REM Sleep may provide a pharmacological platform by itself or in combination with other pharmacological agents to enhance, treat or research declarative (factually-based) memory, non-declarative (relationship-based) memory and learning in patients suffering from, or at risk for Alzheimer's dementia, Parkinson's disease, stroke, and other neurological illness.

Recent research has focused on the role of REM sleep in human mood regulation through neural pathways between the brain cortex (long-term memory storage) and the anatomic amygdala, part of the limbic system or “emotional” center of the brain (Maquet, P. et al. 1996, Functional neuroanatomy of human REM sleep and dreaming”, Nature, Vol. 383: 163-166). Researchers hypothesize that REM sleep, most present in the later parts of a normal, natural sleep cycle, may play a specific role in prioritizing and consolidating emotional memories affecting mood (Wagner et al., 2001, Emotional memory formation is enhanced across sleep intervals with high amounts of rapid eye movement sleep, Learning and Memory, Vol 8: 112-119.). Imaging studies show that the amygdala and the memory storage areas of the brain cortex are active during REM sleep (Nofzinger et al., 1997, Forebrain activation in REM sleep: An FDG PET study, Brain Research, Vol. 770: 192-201). The amygdala, during REM sleep, may prioritize or re-organize emotional memories in the cortex according to “emotional relevance” (Cahill, L. et al, 1995, The amygdala and emotional memory, Nature, Vol. 377, 295-296). The priority and presence of emotional memories affected by REM sleep and associated dreams may affect mood regulation. The present invention of Continuous REM Sleep may provide a pharmacological platform by itself or in combination with other pharmacological agents to treat or study mood-affected disorders such as depression, anxiety, and post-traumatic stress disorder (PTSD).

Much recent sleep research has been focused on the structural and functional brain changes or brain “plasticity” affecting memory, learning and mood regulation. Considerable evidence implicates REM sleep as having a primary role in the brain plasticity of memory consolidation and learning involving declarative memory (factual), non-declarative memory (associative or relationship-based) and emotional memory (emotional relevance) (Walker, M and Stickgold, R. 2006. Sleep, Memory, and Plasticity, Annu. Rev. Psychol., Vol. 57: 139-66). Critical brain areas involved in memory consolidation and learning affected by REM sleep include the amygdala (emotional memory), the hippocampus (short term memory) and multiple areas of the cortex (long-term memory)—specifically the visual cortex and the medial-prefrontal cortex (MPFC) (Walker, M and Stickgold, R., 2004, Sleep-dependent learning and memory consolidation, Neuron, Vol. 44, 121-133).

Timing of REM sleep in memory consolidation of new information accordingly may be desirable. Sleep researchers have proposed the importance of REM sleep “plasticity” windows to describe the timely need for REM sleep in order to create stable long term memories in a natural sleep cycle (Smith, C., 1996, Sleep states, memory processes and synaptic plasticity, Behavioral Brain Research, Vol. 78: 49-56). In one study, REM sleep was found to be only effective in memory consolidation in the last 2 hours of an 8 hour sleep cycle, when REM sleep was most un-fragmented (Stickgold, R. et al., 2000, Visual discrimination task improvement: a multistep process occurring during sleep, J Cog Neurosci, Vol 12: 246-254). By controlling the duration and intensity of Continuous REM Sleep using the process described herein, Continuous REM Sleep can by itself or in combination with other pharmacological agents provide a platform for generating and controlling the REM sleep windows for therapeutic and research applications involving memory and learning and/or mood stabilization.

Neuronal (cellular) links which involve memory and learning functions affected by specific neurotransmitter and receptor pathways are a source of intense research. Strongly suggested by sleep findings are that the neuronal “plasticity” of memory and learning and emotional pathways may be regulated or activated by sleep, and REM sleep, in particular. “Neuronal plasticity” describes a cellular state in which the neuronal cell is modified to become more receptive and active in its functions—in this case the cognitive processes of memory and learning and emotional (mood) regulation. Therefore, Continuous REM Sleep may be used to generate REM sleep-based neuronal plasticity in memory and learning and emotional brain pathways.

Cellular and molecular mechanisms involved in neuronal plasticity rely on synaptically-located neurotransmitter and receptor communication to produce short-term changes (such as protein phosphorylation) in synaptic efficacy and long-term changes (gene transcription and protein synthesis) leading to structural changes in neural synapses involved with memory and learning processes (Tononi, G and Cirelli, C., 2001, Some considerations on sleep and neural plasticity, Archives Italiennes de Biologie, Vol 139: 221-241.). Through methods described in the invention, the timing and intensity of Continuous REM Sleep can be controlled to activate the brain for learning and memory and mood stabilization. The Continuous REM Sleep may serve as a platform by itself or in combination with other cognition enhancing and mood stabilizing drugs (described below) to further enhance neuronal plasticity and augment the efficacy of such neuroactive drugs.

Specific neurotransmitters involved with neuronal synaptic plasticity of memory and learning processes (cognition enhancement) and emotional regulation (mood stabilization) include serotonin, glutamate, acetylcholine, norepinephrine, dopamine, adenosine and cortisol.

Specific drug classes (cognition enhancers and mood stabilizers) that may affect the concentrations of these neurotransmitters or neuronal synaptic efficacy include acetylcholinesterase inhibitors (neostigmine, donepezil, galantamine, rivastigmine), nicotinic agonists (nicotine), serotonin uptake inhibitors (ondansetron), glucocorticoids (dexamethasone), GADA agonists, NMDA antagonists (memantine), NMDA agonists, antipsychotics (haloperidol, buproprion, bromocriptine, selegiline), ampakines (ampalex), calcium channel blockers (nimodipine), excitatory amines (D-cycloserine, glycines), monamine oxidase inhibitors, adenosine antagonists (caffeine), phosphodiesterase inhibitors (propentofylline, papaverine, rolipram), noradrenaline uptake inhibitors (atomoxetine, reboxetine) monamines (norepinephrine, serotonin, dopamine), amphetamines (dexamphetamine), sympathomimetic amines (methylphenidate, ephedrine, modafinil, adrafinil), antidepressants (sertraline, citalopram, aripiprazole, ziprasidone, tinaneptine), cerebral vasodilators (vinpocetine, naftidrofuryl), ergot derivatives (hydergine, nicergoline), pyrrolidinones (piracetam, oxiracetam, aniracetam, nefiracetam and levetiracetam), free radical scavengers (cerebrolysin, idebenone, coenzyme Q10), and neuropeptides (vasopressin, desmopressin, somatostatin, growth hormone, orexins), (Jones et al., 2007, Cognition enhancers, Foresight Brain Science, Addiction and Drugs Project, Vol. 1: 1-44). These cognition enhancement and mood stabilizer drugs and others may be functionally activated or augmented by the methods described herein.

The terms “subject” and “patient” are interchangeable, and are meant to include mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

### Continuous REM Sleep Methodology

In one aspect of the invention, a method is provided for inducing and maintaining Continuous REM Sleep in a subject, comprising: administering to the subject a pharmaceutically effective amount of a first hypnotic anesthetic agent such that the level of consciousness (LOC) of the subject is reduced to lower than 70%, optionally lower than 60%, 50%, 40%, 30%, or 25%, as compared to the LOC measured when the subject is awake. Preferably, a hypnotic or sedative agent is a GABA-A agonist such as propofol which is administered to the subject via bolus injection.

The method may further comprise the step of administering a second hypnotic anesthetic agent to the subject continuously for a period of time at a dose such that the subject remains in hypnosis and the LOC of the subject is maintained during this period of time at about 50-90%, 60-85%, 65-85%, or 70-80% as compared to the LOC measured when the subject is awake. The period of time for a Continuous REM Sleep cycle is preferably 5-240 minutes, 10-120 minutes, or 20-60 minutes, or indefinitely depending on the therapeutic application.

The method may further comprise the step of administering related adjunct agents or medications to the subject as a single injection or continuously as an infusion for a period of time during which the subject remains in hypnosis and the LOC of the subject is maintained during this period of time at about 50-90%, 60-85%, 65-85%, or 70-80% as compared to the LOC measured when the subject is awake.

According to the method, the first and second hypnotic agent may be the same or different. For example, the first and second hypnotic agent can both be propofol. Alternatively, the first hypnotic agent may be propofol, and the second agent may be another, different GABA-A agonist.

According to the method, the related adjunct medications may be ondansetron (serotonin or 5-HT3 uptake inhibitor), dexamethasone (steroid), fentanyl (narcotic), midazolam (benzodiazepine), vasopressors (ephedrine), or local anesthetic (lidocaine). Alternatively, the adjunctive medications may be another serotonin or 5-HT3 uptake inhibitor, steroid, narcotic, benzodiazepine or local anesthetic.

Generally, the present invention describes the production of a Continuous REM Sleep cycle in a subject through the controlled use of a hypnotic pharmaceutical agent such as a GABA-A agonist, followed by rapid recovery to a waking state. The clinical process of a Continuous REM Sleep cycle as described in this invention can be divided into three phases: pre-procedure, Continuous REM Sleep procedure, and post-procedure.

During the pre-procedure phase a patient's history may be obtained prior to the procedure for medical and adverse medication history and recorded. Patient's mental status may be assessed by a qualified mental health professional according to the objectives of the therapy (sleep recovery, memory/learning enhancement or mood stabilization). Oral intake (food, fluid, medications) and restriction guidelines should meet current American Society of Anesthesiologists (ASA) recommendations for sedation anesthesia. Upon check-in at the site of the procedure, the patient is placed in a supine position on a bed or gurney and a peripheral intravenous (IV) line is started with medication ports using a 500 cc normal saline bag kept at a “to keep open” (TKO) rate of infusion. Emergency airway and medications are to be available at bedside.

In one embodiment, during the pre-procedure phase, patient monitors are placed including: 3-lead electrocardiogram (EKG), pulse oximeter, blood pressure cuff and brain function monitor sensors (forehead). Supplemental oxygen is given with nasal cannula or by mask at 2-8 liters per minute. Additionally, capnography may be utilized to monitor for apnea and end tidal carbon dioxide level. Monitoring and oxygen should begin prior to administration of hypnotic agent.

A subject may be monitored for level of consciousness and Continuous REM Sleep using a commercially available brain function monitor. Brain function monitors, developed for the use of anesthesiologists, use specific algorithms to interpret and record cerebral EEG activity to produce level of consciousness indexes that predict depth of anesthesia on a 0 (no cerebral activity) to 100 (completely awake) scale. Specific EEG waveforms and electromyelogram (EMG) information are extracted to maintain sufficient levels of hypnosis (unconsciousness) and risk of awareness and patient movement during surgical anesthesia. Examples of available monitors include bispectral index monitor (also known as a “BIS” monitor) by Aspect, SNAP Index (Stryker) monitor, and Entropy monitor (General Electric). Brain function monitors have also been successfully used to monitor natural sleep EEG waveforms, including REM sleep (Sleigh, J W et al. “The Bispectral Index: A measure of depth of sleep?”, Anesthesia and Analgesia, 1999; Vol. 88: 659-661).

In one embodiment of the Continuous REM procedure phase, the production of a Continuous REM Sleep cycle includes the following four steps: (i) hypnosis and suppression of REM-OFF brain activity; (ii) hypnosis and activation of REM-ON brain activity; (iii) hypnosis and maintenance of REM-ON brain activity; and (iv) cessation of hypnosis and rapid recovery of brain wakefulness.

In a first step (suppression of REM-OFF neurons) of a Continuous REM Sleep cycle, the subject is administered a hypnotic agent. This agent rapidly induces deep hypnosis (loss of consciousness).

In a second step (activation of REM-ON neurons) of a Continuous REM Sleep cycle, the subject is administered the same hypnotic agent at a constant rate (infusion) at a dosage of one-tenth the initial bolus dose. Adjunct brain active agents (serotonin or 5HT-3 uptake inhibitors, steroids, and other agents) are administered (either as a single intravenous injection or as an infusion) at this time.

In a third step (maintenance of REM-ON neurons) of a Continuous REM Sleep cycle, the subject is administered the same hypnotic agent at a constant rate (infusion) to maintain light hypnosis. The infusion rate of the hypnotic agent is titrated in dosage and then maintained at constant rate based on the subject's level of consciousness (based on brain function monitor and clinical signs) and characteristic REM EEG waveform. Hypnotic agent dosage should be adjusted to maintain clinical parameters of Continuous REM Sleep while maintaining hypnosis. Continuous REM Sleep can be maintained indefinitely as long as this step is continued.

In a fourth step (cessation of hypnosis and recovery) of Continuous REM Sleep cycle, the hypnotic agent is discontinued and rapid consciousness and psychological wakeup (clinical signs and brain function monitor) occurs.

In one embodiment, during the post-procedure phase, the patient is recovered, monitored and discharged according the American Society of Anesthesiology (ASA) guidelines for recovery of sedation anesthesia. Mental status and therapeutic objectives (sleep recovery, memory/learning enhancement, or mood stabilization) can be assessed and quantified by a qualified mental health professional. Patient assessment of sleep recovery and dreams are noted, oral fluids given, monitors and IV line removed. If appropriate, care is transferred to another medical professional and/or facility or the patient discharged home after meeting ASA criteria for discharge.

In one embodiment of the present invention, the pharmaceutical agent to be delivered in four steps (above) to the patient is a hypnotic or sedative agent (preferably a GABA-A agonist). The anesthetic agent is delivered to the patient following standard medical procedures for administration of anesthesia. For example, the subject's IV is connected to an adjustable infusion pump set to deliver 2-6 diisopropylphenol (“propofol”).

In one embodiment, the patient as a first step is administered 750 micrograms (mcg) per kilogram (kg) body weight of propofol over 30 seconds to rapidly induce deep hypnosis (loss of consciousness, unresponsive to name) as measured by brain function monitoring and clinical signs. Preferably, the subject is administered between 300 mcg/kg and 2000 mcg/kg propofol to induce hypnosis (unconsciousness). If necessary the propofol dose can be repeated.

As a preferred embodiment, the patient after administration of initial dose of propofol is considered in deep hypnosis when the eyelid reflex is absent and when level of consciousness (LOC) is below 60 percent. Preferentially these parameters are reached within 60 seconds of administration of propofol (bolus) administration.

As a preferred embodiment, as a second step, a propofol infusion is initiated at 75 mcg/kg/minute to activate Continuous REM Sleep. Adjunct medications such as ondansetron (8 mg), dexamethasone (8 mg) and other agents (anti-emetics, local anesthetics, steroids, narcotics, benzodiazepines, neurotransmitters, catecholoamines, etc) are given or are continuously infused at this time as well. Propofol infusion dosage is adjusted preferentially between 25 to 140 mcg/kg/minute to reach light hypnosis (based on LOC) and the emergence of REM clinical signs.

As a preferred embodiment, the patient after initiation of propofol infusion is considered in light hypnosis and successful activation of Continuous REM Sleep activity occurs when subject does not respond to verbal commands, REM-specific EEG wave form (FIG. 5) present, LOC between 60 to 80 percent, phasic (facial) EMG activity (1-3 cycles per second), generalized muscle hypotonia and saccadic ocular muscle (eyelid) movement (0-4 cycles per second) is noted. Preferentially LOC is between 70 to 80 percent and activation of Continuous REM Sleep is within 5 minutes of propofol administration.

As a third step, continuous propofol infusion is maintained at 75 mcg/kg minute to maintain Continuous REM Sleep. Dosage is adjusted preferentially to between 25-140 mcg/kg/minute to maintain light hypnosis and Continuous REM Sleep.

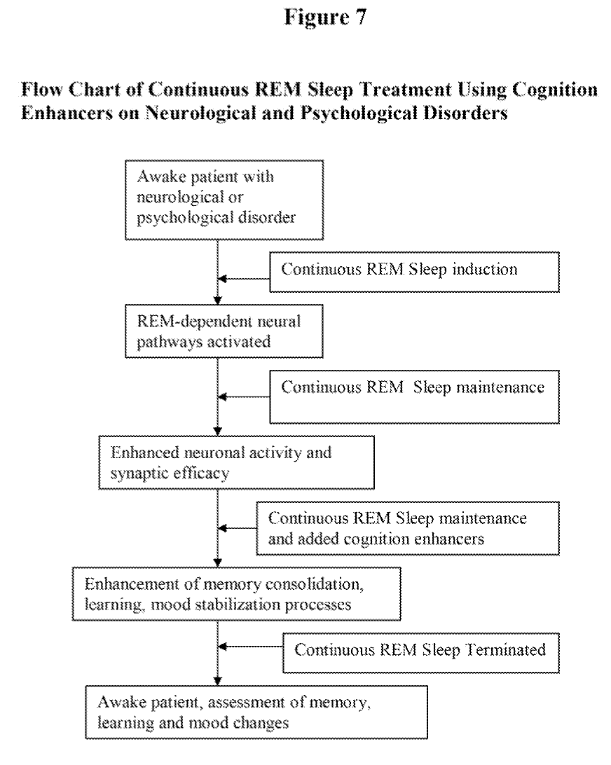

As a preferred embodiment, when the subject maintained on a propofol infusion is successfully maintained in Continuous REM Sleep other potential agents (including but not limited to cognition enhancing and mood stabilizing drugs, see FIG. 7 for example) may be administered at this time. Such cognition enhancers and mood stabilizers or other therapeutic agents may be given prior to, during, or after the initiation of Continuous REM Sleep.

As a preferred embodiment, patient's Continuous REM Sleep cycle does not exceed 240 minutes. In yet another preferred embodiment, the patient's Continuous REM Sleep cycle should be between 5 minutes and 240 minutes. In yet another preferred embodiment, the patient's Continuous REM Sleep cycle should be between 10 minutes and 120 minutes. In the most preferred embodiment, the patient's Continuous REM Sleep cycle should be between 20 minutes and 60 minutes. Although 240 minutes may be a preferred time limit for a patient's Continuous REM Sleep cycle, the cycle may be continued well beyond 240 minutes for considerably longer periods, e.g., indefinitely, for treatment if so desired.

As a fourth step, cessation of propofol infusion at a pre-determined time to allow rapid termination of Continuous REM Sleep and patient wakefulness and response to voice commands within 5 minutes.

In some instances of the invention, once the patient has awakened sufficiently, the supplemental oxygen, IV and monitors are removed. In one embodiment, the person is given oral fluids during recovery. Sleep recovery, dreams, cognition, mood and other psychological, and neurological changes are noted post procedure. After sufficient recovery by ASA standards for sedation anesthesia, he or she is released from care or care is transferred to another medical provider or facility.

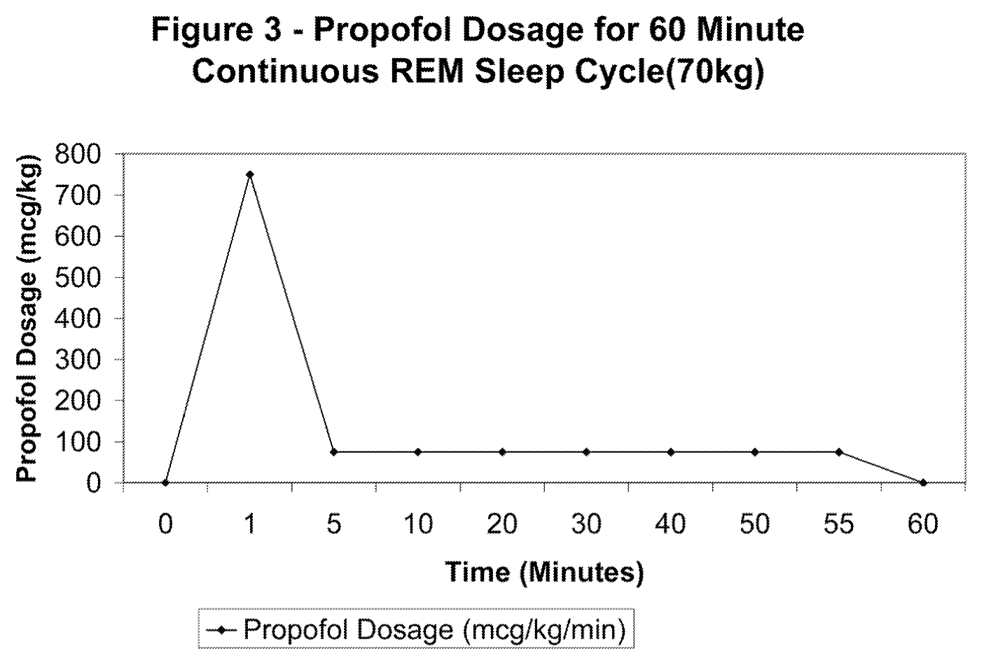

FIG. 3 is a graphical illustration of the infusion rate of the pharmaceutical agent used during a 60 minute Continuous REM Sleep cycle. For example, propofol is administered in a bolus infusion of 750 mcg/kg in the first minute, followed by a maintained dosage of 75 mcg/kg/min for 50 minutes. The infusion rate is then reduced to zero, allowing the patient to awaken shortly.

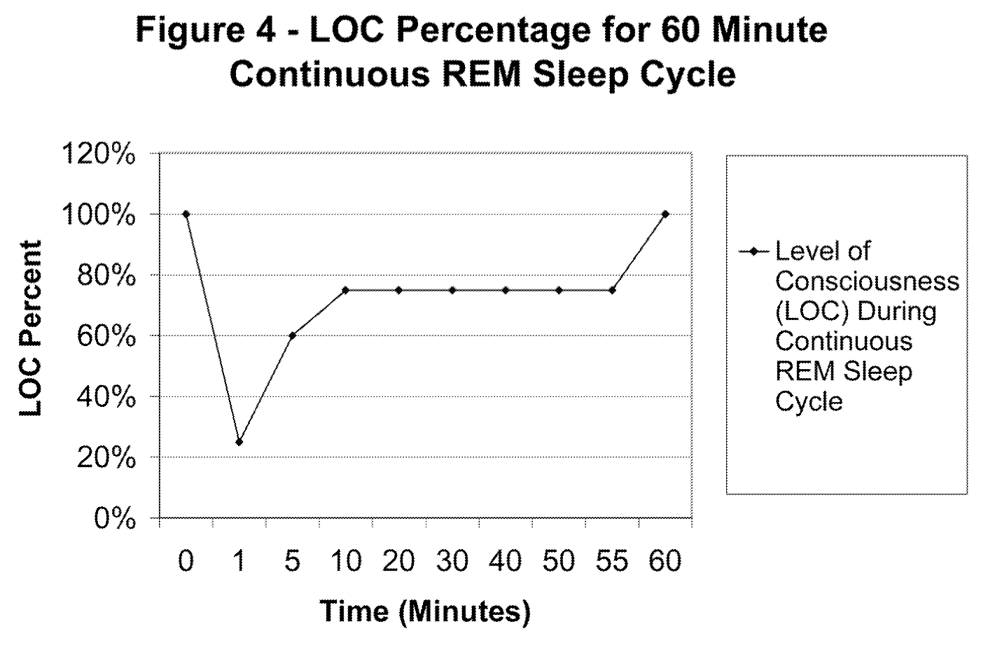

FIG. 4 is a graphical illustration of the approximate level of consciousness of a subject during the Continuous REM Sleep cycle of FIG. 3, and is not meant to be a limiting illustration. As shown therein, the person is fully conscious at the beginning of treatment, but drops quickly to approximately 25% consciousness during the first five minutes of treatment. The person's state of consciousness remains relatively constant for about 45 minutes as a full rate of anesthetic continues to be administered. The infusion rate is terminated after a total of 50 minutes has elapsed, after which the person quickly begins to awaken to a full state of consciousness.

Following a Continuous REM Sleep cycle, patients have reported superlative restfulness (“That was the best night sleep I've ever had.”) along with florid positive dreams (“I was having so many happy dreams.”) and time enhancement (“I've been asleep for days.”). Also significant, post-procedure follow-up reflects immediate mental status benefits (“I felt so good, I went back to work that afternoon.”) with some known chronic insomnia patients reporting better sleep cycles for days afterwards and vivid recall of their dreams experienced under sedation. The present invention may recover many hours and even days of sleep loss in a shortened period of this process, sometimes in less than one hour or even in as short a time as 20 minutes. Additionally, there is evidence based on patient follow-up that the overall sleep cycle (circadian rhythm) is improved, suggesting that hypothalamus may be a significant site of pharmacological action as well. The inventor proposes that these outcomes occur by continuously stimulating the production of un-fragmented REM sleep in the process described in this invention.

GABA is the primary inhibitory transmitter in the brain and maintains a balance between excitation and inhibition of neurons. Three major classes of GABA receptors have been identified: GABA-A, GABA-B and GABA-C receptors. GABA-A and GABA-C receptors are ligand-gated ion channels (LGIC), while GABA-B receptors are G-protein coupled receptors. All known GABA-A receptors contain a plurality of distinct modulatory sites, one of which is the benzodiazepine (BZ) binding site. Other modulatory sites include allosteric sites for picrotoxin, barbiturates, neuroactive steroids and ethanol.

Compounds that selectively bind to the benzodiazepine site, or to other allosteric sites, and enhance the ability of GABA to open GABA-A receptor channels are agonists (or positive allosteric modulators) of GABA receptors. Compounds that interact with allosteric sites but negatively modulate the action of GABA are called inverse agonists (negative allosteric modulators). Inverse agonists diminish the ability of GABA to open receptor channels. A third class of compounds that bind selectively to the benzodiazepine site and yet have little or no effect on GABA activity, but can block the action of GABA-A receptor agonists or inverse agonists that act at this site are referred to as antagonists. Agonists that act at the benzodiazepine site exhibit anxiolytic, sedative, and hypnotic effects, while compounds that act as inverse agonists at this site elicit anxiogenic, cognition enhancing, and proconvulsant effects.

In one embodiment, the present invention should be performed in a controlled medical facility with monitoring and equipment meeting accepted guidelines for sedation anesthesia. Examples of such facilities include hospitals, outpatient treatment centers, surgery centers and mobile units capable of safely delivering sedation anesthesia.

In another embodiment, the present invention can be performed and replicated in research facilities to develop drugs active and efficacious during, prior to, or after a period of REM sleep neuronal plasticity. Such drugs may enhance REM sleep, or alter neural pathways affecting cognition and mood. Such drugs may be also used to alter, change the content, and perception of dream content and intensity produced during REM sleep.

In another embodiment, the present invention can be performed by military medical personnel for use in the field, in a mobile surgical unit, or military hospital unit for rapid and enhanced sleep recovery for sleep distressed or psychologically-impaired military personnel, particularly during combat actions or other time-limited military functions.

In another embodiment, the present invention can be performed at military or veterans hospital or medical facility for the treatment of stress, anxiety and sleep disturbances and disorders in military personnel and veteran patients.

In another embodiment, the present invention can be performed at civilian medical facilities for rapid and enhanced sleep recovery as prescribed by physicians and supervised by medical professionals. Examples of such facilities include but are not limited to sleep diagnostic and treatment centers, hospitals, and outpatient centers and mobile units.

In another embodiment, the present invention can be performed at civilian medical facilities for rapid and enhanced dream sleep therapy at potentially future “dream sleep therapy” centers oriented for the production of therapeutic and stress-relief dreams.

In another embodiment, the present invention can be performed at civilian medical facilities, “cognition” “mood” or “dream” therapy centers, for REM sleep therapy of psychological, psychiatric and neurological disorders by improving learning and memory processes and mood stability. Examples of such facilities include but are not limited to hospitals, medical centers, research laboratories, inpatient and outpatient psychiatric units, outpatient psychotherapy offices and centers, neurological disease (e.g. stroke and Alzheimer's disease) recovery rehabilitation and treatment centers.

In another embodiment, the present invention can use be used to develop the electronic criteria to calibrate, index and map dream content, and memory and learning activity in brain function monitors, polysomnogram monitors, “REM Machines” and other REM detecting devices. Such devices may use EEG waveforms, electromyelogram activity (EMG), ocular muscle movement (eyelid movement), level of consciousness (LOC) levels to detect REM activity induced by the invented method, other methods and occurring naturally. By reliably producing Continuous REM Sleep (using the methodology of Continuous REM Sleep production described herein), EEG, EMG and ocular muscle movements and other REM-sensitive signals can be correlated and indexed with a catalog of specific dream content and cognition and mood activity to develop new REM-detection and interpretation monitoring devices.

In another embodiment, the present invention can be used to develop a catalogued REM sleep EEG database (or “REM Index”) using spectral analysis (fast Fourier transform test, etc.) of REM EEG frequency patterns or “REM Profiles” produced by patients, including but not limited Continuous REM sleep patients. Such a database of REM profiles electronically stored in a REM Index can be clinically used in the electronic interpretation of observed REM. EEG frequency patterns in a treated patient undergoing a sleep state or Continuous REM sleep process to identify specific categories of REM activity. Examples of categories of REM activity include dream content such as positive dreams (pleasant), negative dreams (nightmares) and neutral (task-oriented) dreams and cognition processes such as long term memory or associative memory formation that may be able to be produced and electronically monitored in real time using the present invention. The REM Index can be configured to identify REM frequency pattern changes (for example, between 15-40 Hz) that are correlated with these different processes (i.e., specific dreams, long term memory formation, etc.) for the therapeutic outcomes of mood stabilization and/or cognition enhancement.

The terms “pro-drug” and “prodrug”, which may be used interchangeably herein, refer to any compound which releases an active parent drug in vivo when such prodrug is administered to a mammalian subject. Prodrugs of a compound are prepared by modifying one or more functional group(s) present in the compound in such a way that the modification(s) may be cleaved in vivo to release the parent compound.

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients. In general, the compounds of the invention will be administered in a therapeutically effective amount by any of the accepted modes of administration for agents that serve similar utilities.

In one aspect of the invention, a pharmaceutical composition is provided. The pharmaceutical composition comprises: a dosage form containing a pharmaceutically effective amount of a hypnotic agent which, when administered to a human, stimulates GABA-mediated inhibition of brain neuronal activity for a period of time of 5-240 minutes or indefinitely depending on the therapeutic application

In one aspect of the invention, a pharmaceutical composition is provided. The pharmaceutical composition comprises: a dosage form containing a pharmaceutically effective amount of a hypnotic agent which, when administered to a human, produces GABA-mediated suppression of REM-OFF brain neuronal activity for an unspecified period of time as necessary to activate a REM-ON sleep state for sleep recovery, cognition enhancement and mood stabilization therapy.

The compounds of the invention may be formulated for parenteral administration (e.g., by injection, for example bolus injection or continuous infusion) and may be presented in unit dose form in ampules, pre-filled syringes, small volume infusion or in multi-dose containers with an added preservative. The compositions may take such forms as suspensions, solutions, or emulsions in oily or aqueous vehicles, for example solutions in aqueous polyethylene glycol.

Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

In a preferred embodiment, the pharmaceutical agent according to the present invention is 2-6 diisopropylphenol (also called “2,6 diisopropyl phenol; “propofol”; “diprivan”; “diisopropylphenol”; “2,6-bis(1-methylethyl)phenol”; “disoprofol”; and “milk of anesthesia”). It also is believed that 2-6 diisopropylphenol acts by binding to gamma-aminobutyric acid (GABA-A) receptor A. The chemical formula for 2-6 diisopropylphenol is C₁₂H₁₈O.

The present invention includes prodrugs, analogs and derivatives of propofol, and methods of making the prodrugs, analogs and derivatives, such as those disclosed in US Patent Application Nos. 2007/0185217, 2007;0135390, 2006/0222597, 2006/0205969, 2005/0267169, 2005/0002867, 2004/0265388 and 2004/0220283, which are hereby incorporated by reference. For example, in one embodiment, fospropofol, a water-soluble prodrug of propofol may be used in the present invention. Fospropofol and other aqueous variants of propofol, may circumvent certain disadvantages of a lipid emulsion of propofol, including pain on injection (Picard P, Tramer M R. “Prevention of Pain on Injection with Propofol: A Quantitative Systematic Review”, Anesthesia Analgesia. 2000;90(4):963-969; Nakane M, Iwama H. “A potential mechanism of propofol-induced pain on injection based on studies using nafamostat mesilate”, British Journal of Anaesthesia. 1999;83(3):397-404). Additionally aqueous variants of propofol, such as fospropofol, may more readily be combined with other aqueous medications (related adjunct medications, cognition enhancers, mood stabilizers) into a single syringe or kit than the lipid-based propofol described in the methodology of this invention.

Alternatively, other agents, and in particular, agents that have an agonistic effect on GABA_{A} receptors may be used as a pharmaceutical agent in the present invention alone or in combination. Without limiting the present invention, the pharmaceutical agent may be, for example, benzodiazepines, barbiturates, volatile anesthetics, narcotics, sedative anesthetics, hypnotic anesthetics, antipsychotics, NK1 receptor antagonists, glucocorticoid steroids, local anesthetics, antidepressants, serotonin reuptake inhibitors, GABA II ligands, or mood stabilizers administered in combination as part of the same pharmaceutical composition, as well as to methods in which such active agents are administered separately as part of an appropriate dose regimen designed to obtain the benefits of combination therapy.

In another preferred embodiment, anti-nausea agents that act as 5-HT3 antagonists (serotonin uptake antagonists), such as ondansetron, dolestron and granisetron and corticosteroids, such as dexamethasone, hydrocortisone, methylprednisolone, may be used as adjunct medications in the present invention to produce Continuous REM Sleep.

The appropriate dose regimen described in this invention, the amount of each dose of an active agent administered, and the specific intervals between doses of each active agent will depend upon the subject being treated, the specific active agent being administered and the nature and severity of the specific disorder or condition being treated. Variations may nevertheless occur depending upon the subject being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect.

Exemplary benzodiazepines may include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary barbiturates may include but are not limited to allobarbital, amobarbital, aprobarbital, alphenal, barbexaclone, barbital, brallobarbital and phenobarbital.

Exemplary 5-HT₃ antagonists may include but are not limited to ondasetron, granisetron, dolasetron, tropisetron, palonosetron and ramosetron.

Exemplary narcotics may include but are not limited to fentanyl, remifentanil, alfentanil, sufentanil, morphine, hydromorphone, meperidine, codeine and hydrocodone.

Exemplary antidepressants may include but are not limited to maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics may include but are not limited to clozapine, risperidone, quetiapine, olanzapine, amisulpride, sulpiride, zotepine, chlorpromazine, haloperidol, ziprasidone, and sertindole.

Exemplary mood stabilizers may include but are not limited to Valproic acid (valproate) and its derivative (e.g. divalproex), lamotrigine, lithium, verapamil, carbamazepine and gabapentin.

Exemplary steroids or glucocorticoids may include but are not limited to dexamethasone, hydrocortisone, prednisone, prednisolone, methylprednisolone, betamethasone, triamcinolone, beclometasone, and fludrocortisone.

In certain embodiments, 2-amino-3-(2,6-diisopropylphenoxycarbonyloxy)-propanoic acid or pharmaceutically acceptable salts, or solvates thereof or crystalline forms thereof as disclosed herein, can be used in combination therapy with at least one other therapeutic agent. 2-Amino-3-(2,6-diisopropylphenoxycarbonyloxy)-propanoic acid and the at least one other therapeutic agent can act additively or, in certain embodiments, synergistically. In certain embodiments, 2-amino-3-(2,6-diisopropylphenoxycarbonyloxy)-propanoic acid can be administered concurrently with the administration of another therapeutic agent, such as for example, another sedative, hypnotic agent, or anesthetic agent. In certain embodiments, 2-amino-3-(2,6-diisopropylphenoxycarbonyloxy)-propanoic acid or pharmaceutically acceptable salts, or solvates thereof or crystalline forms can be administered prior or subsequent to administration of another therapeutic agent, such as, for example, another sedative, hypnotic agent, or anesthetic agent.

Pharmaceutical compositions of the present disclosure can include, in addition to one or more compounds of the present disclosure, one or more therapeutic agents effective for treating the same or different disease, disorder, or condition.

Methods of the present disclosure include administration of one or more compounds or pharmaceutical compositions of the present disclosure and one or more other therapeutic agents, provided that the combined administration does not inhibit the therapeutic efficacy of the one or more compounds of the present disclosure and/or does not produce adverse combination effects.

Compounds of the present disclosure and another therapeutic agent or agents can act additively or synergistically. In certain embodiments, compositions of the present disclosure can be administered concurrently with the administration of another therapeutic agent, which can be part of the same pharmaceutical composition as, or in a different composition from, that containing the compounds of the present disclosure. In certain embodiments, compounds of the present disclosure can be administered prior or subsequent to administration of another therapeutic agent. In certain embodiments of combination therapy, the combination therapy comprises alternating between administering a composition of the present disclosure and a composition comprising another therapeutic agent, e.g., to minimize adverse side effects associated with a particular drug. When a compound of the present disclosure is administered concurrently with another therapeutic agent that potentially can produce adverse side effects including, but not limited to, toxicity, the therapeutic agent can advantageously be administered at a dose that falls below the threshold at which the adverse side effect is elicited.

When two or more kinds of drugs selected from the group consisting of sedative antidepressants and antihistamines are used, each dosage of the drugs can be reduced compared to when only one of them is used.

Furthermore, the pharmaceutical composition for preventing or treating sleep, neurological and psychological illnesses or disorders, of the present invention may be jointly used in combination with other active ingredients as long as its advantageous property is substantially not interfered. The other active ingredients, sedative antidepressants and/or antihistamines and compound A may be blended according to a per se known method to give a pharmaceutical composition (e.g., tablets, powders, granules, capsules (including soft capsules), liquids, patches, injections, suppositories, sustained-release preparations, etc.), and the obtained pharmaceutical composition may be administered, or preparations formulated separately may be administered to the same subject simultaneously or at different times in the same way of the preparation of the present invention.

In certain embodiments, a drug can further comprise substances to enhance, modulate and/or control release, bioavailability, therapeutic efficacy, therapeutic potency, stability, and the like. For example, to enhance therapeutic efficacy a drug can be co-administered with one or more active agents to increase the absorption or diffusion of the drug through the gastrointestinal tract, or to inhibit degradation of the drug in the systemic circulation. In certain embodiments, a drug can be co-administered with active agents having pharmacological effects that enhance the therapeutic efficacy of the drug. For example, ephedrine, lidocaine, midazolam, fentanyl, dexamethasone, ondansetron, ketamine may be administered as a adjunct agent with any of the preceding compounds.

In yet another aspect of the invention, a kit is provided for the production of a Continuous REM Sleep Cycle. The kit comprises: a pharmaceutical dosage form containing a pharmaceutically effective amount(s) of a hypnotic and adjunct agents which, when administered to a human, induces and maintains Continuous REM Sleep activity for a specified period of time. The dosage form may be oral or parenteral such as in an injectable formulation suitable for intravenous, intramuscular, subcutaneous administration. For example, the kit may contain a syringe prefilled with an injectable formulation of the hypnotic and adjunct agents in an amount sufficient to induce and maintain Continuous REM Sleep. The kit may also included cognition enhancer and mood stabilizer agents packaged either separately from the hypnotic agents or in combination in the syringe or oral form. The kit may further comprise instructions for how to use the pharmaceutical dosage form for producing Continuous REM Sleep, and/or for treating or preventing psychological, neurological or sleep illnesses or disorders.

### Methods of Use

The present invention describes a novel pharmacological method of producing an unique Continuous REM Sleep state in humans, distinct from fragmented and limited REM sleep that occurs in a natural sleep cycle. This invention, potentially combined with other medications, may provide a platform to develop new research and therapy in many fields treating the human brain.

The present invention describes the production of an unique and specific REM sleep state to treat psychological, neurological and sleep, illnesses or disorders. This is achieved in the present invention by utilizing an active process of REM sleep induction (as opposed to a passive process of natural REM sleep) and the copious production of “REM-like” dream sleep or as defined in this invention as Continuous REM Sleep. The active process of Continuous REM Sleep is preferably produced by a titrated pharmacological infusion.

In one embodiment, the present invention may be used to treat sleep disorders or illnesses and symptoms related to sleep disturbances by providing therapeutic means of rapid and enhanced sleep recovery.

A sleep disorder is a disruptive pattern of sleep that may include difficulty: falling or staying asleep, falling asleep at inappropriate times, excessive total sleep time, or abnormal behaviors associated with sleep. There are more than 100 different disorders of sleeping and waking. They can be grouped into four main categories: problems with staying and falling asleep (e.g., insomnia), problems with staying awake (e.g., sleep state misperception), problems with adhering to a regular sleep schedule (e.g., hypersomnias such as narcolepsy), and sleep disruptive behaviors (e.g., sleep walking). Examples of such sleep disorder include but are not limited to: (1) dyssomnia such as intrinsic sleep disorders (e.g., psychophysiological insomnia), extrinsic sleep disorders, and circadian rhythm disorders (e.g., time zone change syndrome (jet lag), shift-work sleep disorder, irregular sleep wake pattern, delayed sleep-phase syndrome, advanced sleep-phase syndrome, non 24-hour sleep-wake disorder); (2) parasomnias; (3) sleep disorders associated with medical/psychiatric disorders (e.g., chronic occlusive pulmonary disease, Alzheimer's disease, Parkinson's disease, multiinfarct dementia, schizophrenia, depression, anxiety disorders). The sleep disorders can be diagnosed according to the criteria and methods outlined in the Diagnostic and Statistical Manual of Mental Disorders 4.sup.th edition (DSM IV) published by the American Psychiatric Association, Washington, D.C. (1994).

Insomnia has been estimated to affect 40% of North Americans per year (Stoller M K. “Economic effects of insomnia”, Clinical Therapy, September-October 1994; 16(5):873-97). A study by the U.S. National Sleep Foundation and the Gallup Organization involving 1,000 randomly selected Americans revealed that insomnia negatively impacts activities during waking function and effects quality of life (Roth T, Ancoli-Israel S. “Daytime consequences and correlates of insomnia in the United States: results of the 1991 National Sleep Foundation Survey II”, Sleep. May 1, 1999; 22 Suppl 2:S354-8.). Another study involving 261 insomnia sufferers and 101 individuals with no sleep complaints revealed that insomnia significantly impairs quality of life (Zammit G K, Weiner J, Damato N, Sillup G P, McMillan C A. “Quality of life in people with insomnia”, Sleep. May 1, 1999; 22 Suppl 2:S379-85.). Insomnia includes any combination of difficulty with falling asleep, staying asleep, intermittent wakefulness, and early-morning awakening and can lead to the following disorders: delayed sleep phase syndrome, hypnotic dependent disorder, and stimulant dependent sleep disorder.

In one embodiment, the present invention may be used to treat or alleviate the symptoms of psychological disorders and illnesses affecting mood such as anxiety disorders.

Anxiety disorders, panic attacks, and agoraphobia are conditions that occur as a manifestation of primary mood disorders such as depression. Anxiety disorders, as a group, are the most common mental illness in America. More than 19 million American adults are affected by these debilitating illnesses each year. Children and adolescents can also develop anxiety disorders. The five major types of anxiety disorders are identified as: Panic Disorder, Obsessive-Compulsive Disorder, Post-Traumatic Stress Disorder, Generalized Anxiety Disorder and Phobias (including Social Phobia, also called Social Anxiety Disorder). Each anxiety disorder has its own distinct features, but they are all bound together by mood disturbances produced by fear-based anxiety. It is common for an anxiety disorder to accompany depression, eating disorders, substance abuse, or another anxiety disorder.

The compositions, methods, kits and systems can also be used for treating, preventing or alleviating symptoms of psychological disorders, particularly disorders producing mood disturbances, such as those Class 5 mental disorders according to “International Classification of Diseases” (ICD), 9th Revision, Clinical Modification, Seventh Edition, 2007 or ICD-9-CM 2007, including but not limited to: (290) Dementia; (291) Alcohol induced mental disorders; (292) Drug induced mental disorders; (293) Transient mental disorders due to conditions classified elsewhere; (294) Persistent mental disorders due to conditions classified elsewhere; (295) Schizophrenic disorders; (296) Episodic mood disorders; (297) Delusional disorders; (298) Nonorganic psychoses; (299) Pervasive developmental disorders; (300) Anxiety, dissociative and somatoform disorders; (301) Personality disorders; (302) Sexual and gender identity disorders; (303) Alcohol dependence syndrome; (304) Drug dependence; (305) Nondependent abuse of drugs; (306) Physiological malfunction arising from mental factors; (307.4) Specific disorders of sleep of nonorganic origin; (307.41) Transient disorder of initiating or maintaining sleep; (307.42) Persistent disorder of initiating or maintaining sleep; (307.43) Transient disorder of initiating of maintaining wakefulness; (307.44) Persistent disorder of initiating or maintaining wakefulness; (307.45) Circadian rhythm sleep disorder of nonorganic origin; (307.46) Sleep arousal disorder; (307.47) Other dysfunctions of sleep stages or arousal from sleep; (307.48) Repetitive intrusions of sleep; (307.48) Other “Short-sleeper”, subjective insomnia complaint; (307.5) Other and unspecified disorders of eating; (307.81) Tension headache; (308) Acute reaction to stress; (309) Adjustment reaction; (309.81) Posttraumatic Stress disorder; (310) Specific nonpsychotic mental disorders due to brain damage; (311) Depressive disorder, not elsewhere classified; (312) Disturbance of conduct, not elsewhere classified; (313) Disturbance of emotions specific to childhood and adolescence; (314) Hyperkinetic syndrome of childhood; (315) Specific delays in development; (316) Psychic factors associated with diseases classified elsewhere; (317) Mild mental retardation; and (318) Other specified mental retardation (ICD code in parentheses).

In one embodiment, the present invention may be used to treat or alleviate the symptoms of neurological disorders and illnesses affecting memory and learning.

Cognition (memory and learning) neural pathways and the illnesses that affect them primarily involve the brain cortex and their neural networks. The present invention, by activating cortical synaptic plasticity during REM sleep, potentially in conjunction with cognition enhancer drugs may be used to treat memory and learning deficiencies in the presence of damage to degradation of the cortex and their neural networks. Examples of such afflictions include (but not limited to) to Alzheimer's disease, senile dementia, Parkinson's disease, traumatic brain injury, stroke (ischemic, hypoxic and hypoglycemic) and cerebral palsy.

The compositions, methods, kits and systems can also be used for treating, preventing or alleviating symptoms of neurological disorders, such as those Class 6 neurological disorders according to “International Classification of Diseases” (ICD), 9th Revision, Clinical Modification, Seventh Edition, 2007 or ICD-9-CM 2007, including but not limited to: (327) Organic Sleep disorders; (327.0) Organic insomnia; (327.1) Organic hypersomnia; (327.2) Organic sleep apnea; (327.3) Circadian rhythm sleep disorder; (327.4) Organic parasomnia; (327.5) Organic sleep related movement disorders; (330) Cerebral degeneration disease usually manifest in childhood; (331) Other Cerebral degenerations; (331.0) Alzheimer's disease; (331.2) Senile degeneration of brain; (331.7) Cerebral degeneration in diseases classified elsewhere; (332) Parkinson's disease; (346) Migraine headaches; (347) Cataplexy and narcolepsy (ICD code in parentheses).

“REM Machines”: Electronic Systems for the Production. Maintenance, and Interpretation of Continuous REM Sleep and Other REM Sleep

In another aspect of the invention, a new monitoring system is provided for inducing, maintaining, and interpreting electronic data from a Continuous REM Sleep cycle or other REM sleep produced by another method or naturally, according to therapeutic goals of the practitioner. This system is designed to be used with standard monitoring (EKG, pulse oximetry, blood pressure and capnometry) set aside by American Society of Anesthesiologists (ASA) guidelines for sedation anesthesia monitoring.

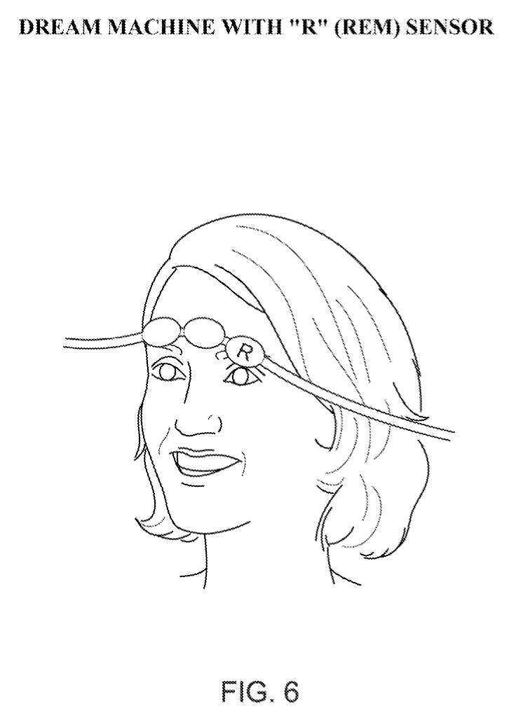

In one embodiment, the system (“REM Machine”) is provided for identifying the specific presence and rating the relative strength of Continuous REM Sleep or other REM sleep. This system may utilize the combined information of the following four variables: level of consciousness (LOC), phasic electromyelogram (EMG) activity (cycles per second), ocular muscle (OM) movement (cycles per second), and presence of specific REM electroencephalogram (EEG) waveforms. LOC parameters would be used to guide anesthesia delivery to induce and maintain Continuous REM or other REM activity. The LOC, EMG, OM and EEG variables would be used to identify the production and relative strength of Continuous REM Sleep or other REM production. For example, by gathering this information from a monitoring strip, such as a “R” (REM) sensor, placed on the patient's forehead at the level of a single eyelid (FIG. 6), this monitoring system can use a processor, e.g., computer, utilizing an electronic algorithm that combines these REM sleep variables (any two, preferably all four of LOC, EMG, OM, and EEG) into a single “REM Score”.

Alternatively, in this and other variations described herein, the system may utilize just a few of these four variables, e.g., two of the four variables, in calculating the composite REM Score. Moreover, such a system may also be utilized with patients who are not only induced into a Continuous REM Sleep state, as described above, but may also be utilized with other patients who are not induced but rather in other states of consciousness such as natural sleep.

Although the REM Machine is described for use with Continuous REM activity (which is induced utilizing methods described herein), it is understood that such a REM Machine may be utilized for the detection and identification of other REM activity (natural or otherwise which may be induced via alternative methods) and any other sleep states to the extent that the patient cycles in and out of a REM state.

Moreover, such a system may allow a practitioner to control or affect any number of parameters in treating a patient. For example, such a system may allow for the practitioner to adjust any number of medications (e.g., hypnotics and/or adjunct meds and/or possibly cognition and/or mood enhancing drugs) to achieve a targeted REM Score. It may also allow for the practitioner to measure REM sleep wave frequency (“REM Intensity”) as a proxy for intensity of REM sleep produced. Such a system also allows for the documentation of REM sleep time as time spent in a cerebral neuronal plasticity state as well as providing copies or a receipt for the patient and charting of such activity. In one example, the REM Intensity of a Continuous REM sleep state induced in a patient can be determined, at least in part, by the average frequency of REM sleep. For instance, if the average frequency measured over a period of time, e.g., a one hour period, of Continuous REM sleep is 20 Hz in a first patient A and 40 Hz in a second patient B, then patient B may be said to have received a REM Intensity which is twice that of patient A and theoretically twice as much REM sleep.

Additionally, the system may allow for real-time identification of categories of REM activity or REM profiles stored in an electronic “REM Index”, including dream content or cognition activity. For example, if the generation of a long term memory in a patient is determined to exhibit exemplary REM EEG frequencies alternating between 30 Hz for one minute followed by 15 Hz for five minutes in a repetitive manner, then the detection of such a pattern may be indicative that the patient is forming a long term memory. Other frequency patterns may, of course, be correlated to other identified cognitive activities. By utilizing the recognition of particular REM EEG frequency patterns or REM Profiles, the system may be preset to identify particular REM Profiles and to indicate to the practitioner, e.g., via an alarm, to allow the practitioner to adjust medications to achieve a particular desired dream or cognitive effect.

In another embodiment, the system will be configured to set limits for LOC for induction, maintenance and termination of a Continuous REM Sleep or other REM sleep. For example only, the system uses visual or auditory signals to notify the user when LOC<60 (for induction of REM), LOC is between 70 to 80 (presence of REM activity), and LOC>80 (an awake state). The system can prompt for the addition of adjunct medications used in the Continuous REM Sleep or REM process or other neuroactive medications including cognition enhancers and mood stabilizers.

In another embodiment, the system will be configured to measure phasic electromyelogram (EMG) activity. During REM sleep, ocular muscles are active, unlike other muscle systems in the body, which are inactive and hypotonia. In this invention, this system will measure the electromyelogram activity of peri-ocular muscles which are oscillate at a frequency (1-3 cycles per second) similar to observed ocular muscle (eyeball) movement. The presence of these peri-ocular EMG oscillations and its frequency will be incorporated into an EMG variable value to be added into the single “REM Score”.

In another embodiment, the system should provide for a new EEG lead system measuring saccadic (rhythmic) eyelid displacement secondary to underlying ocular muscle (eyeball) movement (see FIG. 6) to measure an OM value (0-4 cycles per second). Current brain function monitors used in anesthesia measure EEG activity using specific algorithms to determine level of consciousness (LOC) along with gross EMG values to predict likelihood of patient movement during surgery. In this invention, a specific sensor would be placed directly on the eyelid to measure the frequency of eye movement during Continuous REM Sleep or other REM sleep (FIG. 6). The active eyelid movement seen during Continuous REM Sleep (approximately 0-4 cycles per second), caused by underlying eyeball movement, will be tabulated as an ocular muscle (OM) variable value, which will be added into the single REM Score

In another embodiment, the system will identify REM EEG waveforms. REM EEG waveforms have a REM-specific EEG pattern of high frequency, low amplitude, desynchronized “saw tooth” EEG waves. The presence of these REM signature, “sawtooth” waves (see FIG. 5) will be electronically assessed in this system as an REM EEG value and added into the single REM Score.

In another embodiment, the system should be able to monitor Continuous REM Sleep production or other REM sleep by monitoring the REM Score and specific changes in LOC, EMG, OM, and EEG variables. This will allow the practitioner to adjust the pharmacological agent administration to achieve and maintain Continuous REM Sleep or other REM sleep The system may have adjustable audible and visual alarms to identify when the REM Sleep Score reaches an acceptable threshold score of Continuous REM Sleep or other REM sleep activity and a lower limit alarm to identify when the REM Score is below the acceptable threshold of Continuous REM Sleep or other REM sleep activity.

In another embodiment, the system will track the intensity of Continuous REM Sleep or other REM sleep, as “REM Intensity” by measuring the average frequency of REM EEG waves in cycles per second or Hertz (Hz). Typical REM EEG waves are a fast “theta” frequency of greater than 15 cycles per second or 15 Hz, as compared to the relatively slow (delta) frequency of 1-4 Hz for slow wave sleep. Continuous REM EEG waves are uniformly “theta” frequency (by observation of the inventor), and range from 15-40 Hz. The monitoring system can be configured to alert practitioner with visual and auditory alarms when REM Intensity Score reaches preset or desired levels.

In another embodiment, the system will create a separate “REM Index” by using electronic spectral analysis of sleep state EEG data (i.e. Continuous REM EEG data), such as fast Fourier transform test (FFT), to recognize signature variations in REM EEG frequencies produced by specific REM activity such as specific dream types or cognition processes. Continuous REM EEG wave frequencies range from 15-40 Hz and are uniformly in the fast theta (>15 Hz) frequency. For example, documented specific dream types (such as positive, negative, neutral dreams) and cognition processes (such as long term memory, declarative memory, non-declarative memory formation) can be electronically catalogued and stored in the system as a REM Profile for the therapeutic purposes of real time electronic identification of signature variations in theta EEG frequencies produced in a REM sleep state, such as Continuous REM Sleep. Such a system could also measure other sleep state activity (if at all present) and time ratios and sequences between sleep stages.

In another embodiment, the system will interpret specific dream content by comparing real time theta EEG wave frequency patterns to prior REM Profiles of similar dream content electronically stored in a REM Index. REM index of dream content can be produced by using the present invention of Continuous REM Sleep or other REM sleep using another method or produced naturally to create a library of REM Profiles, which include at least dream-based signature REM EEG frequency patterns. The REM Index can be used to electronically interpret REM EEG patterns and identify real time dream content in patients undergoing Continuous REM Sleep or other REM sleep therapy. Monitoring system can be configured to alert practitioner with visual and auditory alarms when specific dream types are identified, by comparison to pre-existing REM Profiles stored in a REM. Index, by preset or desired dream types (for example positive, negative or neutral dreams).

In another embodiment, the system will interpret specific cognition (memory and learning) activity by comparing theta EEG wave frequency patterns to prior REM Profiles of similar cognition activity electronically stored in a REM Index. REM Indexes of cognition processes can be produced by using the present invention of Continuous REM Sleep to create a library of cognition-based signature REM Profiles, of at least the REM EEG frequency patterns. The REM Index can be used to electronically interpret real time EEG patterns in patients undergoing Continuous REM Sleep therapy or other REM therapy. Monitoring system can be configured to alert practitioner with visual and auditory alarms when specific cognition processes are identified, by comparison to pre-existing REM Profiles stored in a REM Index, by preset or desired cognition activity (for example long term memory, declarative or non-declarative memory formation).

Optionally, the system may track the time spent in Continuous REM Sleep or other REM sleep, and provide an audible and visual alarm to notify the practitioner when the prescribed time, REM Score, REM Intensity, REM Profile of a specific cognition process, or dream type (identified from REM index) is reached. The system will allow for electronic storage and printout of patient record of Continuous REM Sleep or other REM therapy including a summary of date, patient identifiers (name, SSN, etc.), patient diagnosis, time spent in Continuous REM Sleep or other sleep state, medications used, REM Score, REM Profile (dream type or cognition activity), summaries of LOC, EMG, OM and EEG variable values, REM Intensity (average cycles per second) and immediate post-procedure assessment of therapeutic goals including clinical assessment of mental status, sleep recovery, described dreams, mood and/or cognition changes.

The system may further comprise: the compositions (e.g., anesthetics, GABA agonists, propofol, propofol analogs or prodrugs, benzodiazepines, barbiturates, narcotics, nonbenzodiapines sedatives, psycholeptics, nitrous oxide and volatile anesthetic gases) of the invention and instructions for use. The kit may further contain a least one additional reagent, or one or more additional compounds of the invention (e.g., lidocaine, fentanyl, dexamethasone and ondansetron, which may act as adjuvants). The kit may further contain cognition enhancers and/or mood stabilizer agents either in combination with hypnotic agents, adjunct medications, or separately packaged for administration. Furthermore, the system may incorporate a separate or integrated vital sign monitoring apparatus. The system may also further include a printer and/or memory or other data storage media (CD-ROM, computer software, DVD, or other forms of computer-readable medium) instructions for how to use the system to carry out the procedures or methods according to the present invention.

In another aspect of the invention a method for research and drug development is described here. The generation of Continuous REM Sleep in this invention may produce neuronal plasticity by changing the synaptic efficacy of neurons involved in neural pathways involved in sleep, cognition, and mood processes. The methodology and medications described in this invention may allow researchers to identify REM-specific molecules and receptors and develop new drug therapy in sleep, circadian rhythm abnormalities, psychological, and neurological disorders and illnesses.

In yet another aspect of the invention, a method for conducting a clinical business is provided. The method comprises providing a hypnotic agent; and administering the hypnotic agent to a subject in need of treatment, prevention or alleviation of sleep-related or sleep-affected illnesses or disorders. The business method may further include advertising the use of a hypnotic agent for dream therapy, treatment, prevention or alleviation of sleep-related or sleep-affected diseases or disorders, in printed or recorded media and/or on the Internet. Such advertisements may take the form of “dream sleep,” “dream therapy”, “dream anesthetic”, “super sleep”, “hyper dream”, “hyper sleep”, “REM sleep”, “memory sleep”, “cognition sleep” or some other marketing description of the present invention.

The method may further include providing the hypnotic agent, related adjunct medications, other agents and systems, kits, and instructions or training for use of the agent(s) and processes to a physician, health care provider or organization (such as the military) for administration to a subject (patient) in need of treatment, prevention or alleviation of sleep, cognition, or mood-based illnesses or disorders. Such methods may constitute forming an education or consulting entity to either provide the information or directly perform the present invention to interested parties. Such instructions for use of the hypnotic agent, adjunct agents, and cognition enhancers and/or mood stabilizers can include the methods and procedures described herein. The method may optionally include billing the patient or the patient's insurance provider. The method may also include providing kits disclosed herein to a physician or health care provider.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLE 1

Patient A is a 59-year old female who is diagnosed with chronic depression and post-traumatic stress disorder and has been on disability for 10 years. She has suffered from repeated poor quality sleep and often has night terrors or nightmares that are triggered by a variety of environmental stressors.

Patient A received Continuous REM Sleep of approximately 1 hour, as described by to the present invention, while undergoing surgery. Briefly, propofol was administered to her via a bolus injection at a dose of 50 mg (700 mcg/kg) over 30 seconds; and then a continuous infusion of propofol at 120 mcg/kg/minute was maintained for 45 minutes as titrated to clinical signs of REM sleep (hypnosis and ocular muscle movement). Adjunct medications used in her care included lidocaine, midazolam, fentanyl, dexamethasone and ondansetron. After the procedure, Patient A reported having wonderful “dreams” and says she felt extremely rested as if she had slept well for “days”. She reported profuse dreaming about gardening occurring during the procedure. Subsequently on follow-up, Patient A reported lack of nightmares that had previously characterized her chronic depression and post-traumatic stress disorder, as well as improved ease of sleeping post-operatively for three days.

What is claimed is:

1. A method of inducing and maintaining a Continuous REM Sleep cycle in a subject, comprising:
administering to the subject a pharmaceutically effective amount of a first hypnotic agent such that the level of consciousness (LOC) of the subject is reduced to lower than 70%, 60%, 50%, 40%, 30%, or 25%, as compared to the LOC measured when the subject is awake, thereby inducing a Continuous REM Sleep state of the subject.

2. The method of claim 1, wherein the first hypnotic agent is administered to the subject via bolus injection.

3. The method of claim 1, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake, thereby maintaining a Continuous REM Sleep state of the subject.

4. The method of claim 3, wherein the first and second hypnotic agents are the same.

5. The method of claim 3, wherein the first and second hypnotic agents are different.

6. The method of claim 3, wherein the first or the second hypnotic agent is a GABA-A agonist.

7. The method of claim 3, wherein the first or the second hypnotic agent is propofol, or its prodrug, metabolite, analog, or derivative.

8. The method of claim 3, wherein the first and the second hypnotic agents are propofol, or its analog, metabolite, prodrug or derivative.

9. The method of claim 3, wherein the second hypnotic agent is administered to the subject continuously at a dose such that the subject remains in hypnosis and the LOC of the subject is maintained at 70% to 80% with electromyelogram (EMG) activity present, ocular muscle movement and REM-characteristic electroencephalogram (EEG) waveform.

10. The method of claim 3, further comprising: discontinuing the administration of the second hypnotic agent to awake the subject such that the LOC of the subject is greater than 85% as to the LOC measured when the subject is awake.

11. The method of claim 3, wherein the first or second hypnotic agent is selected from the group consisting of benzodiazepines, cyclopyrrones, neurosteroids, barbiturates, etomidate, propofol, narcotics, fospropofol and ketamine.

12. The method of claim 1, wherein the first hypnotic agent is propofol and administered at a dose of between about 300 mcg and 2000 mcg/kg.

13. The method of claim 3, wherein the hypnosis is measured as a LOC score of less than 60%.

14. The method of claim 3, wherein the hypnosis is measured as a LOC score between 60% and 80%.

15. The method of claim 3, wherein the first and the second hypnotic agents are both propofol and administered at an effective dose such that induction of the Continuous REM Sleep is achieved in less than 5 minutes.

16. The method of claim 15, wherein the maintenance of the Continuous REM Sleep lasts for about 5 to 240 minutes, but not more than 240 minutes.

17. The method of claim 15, wherein the maintenance of the Continuous REM Sleep lasts for more than 240 minutes.

18. The method of claim 3, wherein the second hypnotic agent is propofol and the maintenance of the Continuous REM Sleep is measured as a LOC score of between 70% and 80% with EMG activity present, ocular muscle movement and REM-characteristic EEG waveform.

19. The method of claim 3, wherein the second hypnotic agent is propofol and administered in continuous infusion between about 25 mcg to about 140 mcg/kg/min for about 5 to 240 minutes for the maintenance of Continuous REM Sleep.

20. The method of claim 19, further comprising: adjusting the dose of propofol so as to maintain hypnosis and Continuous REM Sleep clinical signs.

21. The method of claim 3, wherein the Continuous REM Sleep state of the subject is determined by monitoring brainwave activity in the subject using a brain function monitor.

22. The method of claim 1, wherein administering to the subject further produces a state in the subject selected from the group consisting of dreams, neuronal plasticity, memory and learning enhancement, mood stabilization, and sleep recovery.

23. A method for inducing and maintaining a Continuous REM Sleep in a subject, comprising: administering to the subject a pharmaceutically effective amount of a hypnotic agent such that GABA-mediated inhibition of brain neuronal activity results in stimulation of REM neuronal activity for a period of time of 5 to 240 minutes.

24. The method of claim 23, wherein the maintenance of the Continuous REM Sleep lasts for more than 240 minutes.

25. The method of claim 23, wherein the hypnotic agent is propofol and administered at a dose of between about 300 mcg and 2000 mcg/kg.

26. The method of claim 23, wherein the subject is a human.

27. A pharmaceutical composition, comprising: a dosage form containing a pharmaceutically effective amount of a hypnotic agent which, when administered to a human, produces GABA-mediated inhibition of brain neuronal activity for a period of time of at least 5 minutes.

28. The pharmaceutical composition of claim 27, where the period of time is between 5 to 240 minutes.

29. The pharmaceutical composition of claim 27, where the period of time is more than 240 minutes.

30. The pharmaceutical composition of claim 27, wherein the dosage form is for oral or parenteral administration.

31. The pharmaceutical composition of claim 27, wherein the dosage form is in an injectable formulation suitable for intravenous, intramuscular, or subcutaneous administration.

32. A method for preventing, alleviating symptoms of, or treating a psychological or neurological condition of a subject, comprising:
administering to the subject a pharmaceutically effective amount of a first hypnotic agent such that the level of consciousness (LOC) of the subject is reduced to lower than 70%, 60%, 50%, 40%, 30%, or 25%, as compared to the LOC measured when the subject is awake.

33. The method of claim 32, wherein the first hypnotic agent is administered to the subject via bolus injection.

34. The method of claim 32, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake.

35. The method of claim 34, wherein the first or second hypnotic agent is selected from the group consisting of benzodiazepines, cyclopyrrones, neurosteroids, barbiturates, etomidate, propofol, narcotics, fospropofol, and ketamine.

36. The method of claim 34, wherein the first and second hypnotic agents are propofol, its prodrug, metabolite, analog, or derivative.

37. The method of claim 32, wherein the psychological or neurological condition is selected from the group consisting of depression and mood disorders, anxiety disorders, chronic fatigue syndrome, sleep walking, sleep disruptive behaviors, insomnia, sleep and waking disorders, sleep disturbances, hypomania, cyclothymia, bi-polar disorders, hyperactivity, attention deficit disorder, tension headaches, premenstrual syndrome (PMS), premenstrual dysphoric disorder (PMDD), agoraphobia, and Class 5 and Class 6 mental and neurological disorders.

38. The method of claim 34, further comprising: administering to the subject or person a neurological agent that is different from the first or second hypnotic agent.

39. The method of claim 38, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

40. A method for achieving a circadian rhythm phase-shifting effect in a person, or treatment, alleviation of symptoms, or prevention of circadian rhythm disorders, comprising:
administering to the person a pharmaceutically effective amount of a first hypnotic agent such that the level of consciousness (LOC) of the subject is reduced to lower than 70%, 60%, 50%, 40%, 30%, or 25%, as compared to the LOC measured when the person is awake.

41. The method of claim 40, wherein the first hypnotic agent is administered to the person via bolus injection.

42. The method of claim 40, further comprising:
administering a second hypnotic agent to the person continuously at a dose such that the person remains in hypnosis and the LOC of the person is maintained during this period of time at about 50-90%, 60-85%, 65-85%, or 70-80% as compared to the LOC measured when the person is awake.

43. The method of claim 42, wherein the first or second hypnotic agent is selected from the group consisting of benzodiazepines, cyclopyrrones, neurosteroids, barbiturates, etomidate, propofol, narcotics, fospropofol and ketamine.

44. The method of claim 42, wherein the first and second hypnotic agents are propofol, its prodrug, metabolite, analog or derivative.

45. The method of claim 42, further comprising: administering to the subject or person a neurological agent that is different from the first or second hypnotic agent.

46. The method of claim 45, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

47. A method for treating a stress related disorder of a subject, comprising:
administering to the subject a pharmaceutically effective amount of a first hypnotic agent such that the level of consciousness (LOC) of the subject is reduced to lower than 70%, 60%, 50%, 40%, 30%, or 25%, as compared to the LOC measured when the subject is awake.

48. The method of claim 47, wherein the first hypnotic agent is administered to the subject via bolus injection.

49. The method of claim 47, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake.

50. The method of claim 49, wherein the first or second hypnotic agent is selected from the group consisting of benzodiazepines, cyclopyrrones, neurosteroids, barbiturates, etomidate, propofol, narcotics, fospropofol and ketamine.

51. The method of claim 49, wherein the first and second hypnotic agents are both propofol, its prodrug, metabolite, analog or derivative.

52. The method of claim 47, wherein the stress related disorder is selected from the group consisting of post-traumatic stress disorder (PTSD), Gulf War Syndrome, chronic fatigue syndrome, fibromyalgia, somatic, affective, and depressive disorders.

53. The method of claim 45, further comprising: administering to the subject or person a neurological agent that is different from the first or second hypnotic agent.

54. The method of claim 53, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

55. The method of claim 54, wherein the neurological agent is selected from the group consisting of ondansetron, dexamethasone, lidocaine, fentanyl, and midazolam.

56. A kit, comprising: a pharmaceutical dosage form containing a pharmaceutically effective amount of a hypnotic agent which, when administered to a human, produces GABA-mediated inhibition of brain neuronal activity for a period of time of at least 5 minutes.

57. The kit of claim 56, wherein the period of time is between 5 to 240 minutes.

58. The kit of claim 56, wherein the period of time is more than 240 minutes.

59. The kit of claim 56, wherein the dosage form is oral or parenteral.

60. The kit of claim 56, wherein the kit contains a syringe prefilled with an injectable formulation of the hypnotic agent in an amount sufficient to induce and/or maintain Continuous REM Sleep.

61. The kit of claim 56, further comprising a neurological agent that is different from the hypnotic agent.

62. The kit of claim 61, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

63. The kit of claim 61, wherein the neurological agent is selected from the group consisting of ephedrine, lidocaine, midazolam, fentanyl, dexamethasone and ondansetron.

64. The kit of claim 56, further comprising: instructions for how to use the pharmaceutical dosage form for producing Continuous REM Sleep, and/or for treating or preventing a psychological or neurological condition, a stress-related disorder, or a sleep-related or sleep-affected neurological disorder.

65. A system for identifying the presence and strength of REM sleep in a subject, comprising:
a monitoring device for measuring at least two of the four variables i) level of consciousness (LOC), ii) electromyelogram (EMG) activity, iii) ocular muscle (OM) movement, and iv) presence of specific “REM-like” electroencephalogram (EEG) waveforms;
a processor in communication with the monitoring device for calculating a REM Score based on the at least two of the i)-iv) variables; and
an indicator for identifying the presence and strength of REM sleep in a subject based upon the calculated REM Score.

66. The system of claim 65, wherein the monitoring device comprises a REM monitoring strip positionable upon a forehead of the subject.

67. The system of claim 65, wherein the indicator comprises an adjustable audible and/or visual alarm configured to identify when the REM Score reaches a predetermined threshold score of REM sleep activity.

68. The system of claim 67, wherein the indicator further comprises a lower limit alarm to identify when the REM Score is below the predetermined threshold of REM sleep activity.

69. The system of claim 65, wherein the processor is configured to track a time the subject has spent in REM sleep.

70. The system of claim 65, wherein the processor is configured to calculate a REM Intensity score.

71. The system of claim 70, wherein the REM Intensity comprises a frequency of REM EEG waveforms in cycles per second.

72. The system of claim 65, further comprising memory for storing a record of the subject's REM sleep parameters.

73. The system of claim 72, wherein the parameters are selected from the group consisting of a summary of date, patient identifiers, patient diagnosis, time spent in REM sleep, REM scores, data indicative of LOC, EMG, OM and EEG waveform and variable scores, post-procedure assessment of dreams, data indicative of patient mood changes, data indicative of sleep recovery, cognition and mood enhancement and other therapeutic goals.

74. A method of monitoring a subject undergoing REM Sleep, comprising:
administering to the subject a pharmaceutically effective amount of a first hypnotic agent such that the level of consciousness (LOC) of the subject is reduced to lower than 70% as compared to the LOC measured when the subject is awake such that a REM sleep state is induced in the subject;
measuring in the subject at least two of four variables including i) level of consciousness (LOC), ii) electromyelogram (EMG) activity, iii) ocular muscle (OM) movement, and iv) presence of specific electroencephalogram (EEG) waveforms indicative of a REM-like state; and
maintaining the REM Sleep state in the subject for a predetermined period of time.

75. The method of claim 74, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake, thereby maintaining a REM sleep state of the subject.

76. The method of claim 75, wherein the first or the second hypnotic agent is a GABA-A agonist.

77. The method of claim 75, wherein the first or the second hypnotic agent is propofol, or its prodrug, metabolite, analog, or derivative.

78. The method of claim 75, wherein the first and the second hypnotic agents are propofol, or its prodrug, metabolite, analog or derivative.

79. The method of claim 75, wherein the first or second hypnotic agent is selected from the group consisting of benzodiazepines, cyclopyrrones, neurosteroids, barbiturates, etomidate, propofol, narcotics, fospropofol and ketamine.

80. The method of claim 75, further comprising: administering to the subject or person a neurological agent that is different from the first or second hypnotic agent.

81. The method of claim 80, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

82. The method of claim 80, wherein the neurological agent is selected from the group consisting of ondansetron, dexamethasone, lidocaine, fentanyl, and midazolam.

83. The method of claim 75, wherein the second hypnotic agent is administered to the subject continuously at a dose such that the subject remains in hypnosis and the LOC of the subject is maintained at 70% to 80% with electromyelogram (EMG) activity present, ocular muscle movement and REM-characteristic electroencephalogram (EEG) waveform.

84. The method of claim 74, wherein measuring further comprises calculating a REM Score based on the at least two of the i)-iv) variables.

85. The method of claim 84, further comprising identifying the presence and strength of a REM Sleep in a subject based upon the calculated dream REM Score.

86. The method of claim 84, further comprising indicating when the REM Score falls below a predetermined threshold value.

87. The method of claim 74, wherein measuring comprises positioning a monitoring strip upon a forehead of the subject.

88. The method of claim 74, wherein maintaining comprises maintaining sufficient REM Score parameters for REM Sleep.

89. The method of claim 74, further comprising terminating the REM sleep state.

90. The method of claim 83, further comprising calculating a REM Score based on the at least two of the i)-iv) variables.

91. The method of claim 90, further comprising adjusting an amount of the first hypnotic agent and/or second hypnotic agent to adjust the REM Score.

92. The method of claim 74, wherein measuring further comprises measuring a REM sleep wave frequency or REM Intensity in cycles per second.

93. The method of claim 92, further comprising indicating when the REM Intensity surpasses at least one preset level.

94. The method of claim 74, further comprising documenting the REM sleep state

95. The method of claim 74, wherein measuring further comprises measuring a duration of the REM-like state.

96. A method of therapy via production of Continuous REM Sleep, comprising:
administering to a subject a pharmaceutically effective amount of a first hypnotic agent such that the level of consciousness (LOC) of the subject is reduced to lower than 70% as compared to the LOC measured when the subject is awake, thereby inducing a Continuous REM Sleep state in the subject; and
administering to the subject a pharmaceutically effective amount of a cognition enhancing agent or mood stabilization agent while the Continuous REM Sleep state is maintained such that synaptic plasticity in neurons of the subject are created or enhanced.

97. The method of claim 96, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake, thereby maintaining a Continuous REM Sleep state of the subject.

98. The method of claim 97, wherein the first or the second hypnotic agent is a GABA-A agonist.

99. The method of claim 97, wherein the first or the second hypnotic agent is propofol, or its prodrug, metabolite, analog, or derivative.

100. The method of claim 97, wherein the first and the second hypnotic agents are propofol, or its analog, metabolite, prodrug or derivative.

101. The method of claim 97, wherein the second hypnotic agent is administered to the subject continuously at a dose such that the subject remains in hypnosis and the LOC of the subject is maintained at 70% to 80% with electromyelogram (EMG) activity present, ocular muscle movement and REM-characteristic electroencephalogram (EEG) waveform.

102. The method of claim 97, wherein the first or second hypnotic agent is selected from the group consisting of benzodiazepines, cyclopyrrones, neurosteroids, barbiturates, etomidate, propofol, narcotics, fospropofol and ketamine.

103. The method of claim 96, further comprising completing administration of the cognition enhancing agent whereby sleep recovery in the subject is facilitated.

104. The method of claim 96, further comprising completing administration of the cognition enhancing agent whereby memory and learning in the subject is enhanced.

105. The method of claim 104, wherein memory consolidation, declarative memory, non-declarative memory, and emotional memory are enhanced.

106. The method of claim 96, further comprising completing administration of the cognition enhancing agent whereby a mood of the subject is stabilized.

107. The method of claim 96, wherein the cognition enhancing or mood stabilizing agent is selected from the group consisting of acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

108. The method of claim 96, further comprising controlling a duration and/or intensity of the Continuous REM Sleep state such that the cognition enhancing agent or mood stabilization agent is administered to the subject prior to, during, or after initiation of the Continuous REM Sleep state.

109. A method of correlating a dream state and/or cognition process of a subject undergoing REM Sleep, comprising:
inducing the subject into a REM Sleep state by administering a pharmaceutically effective amount of a first hypnotic agent such that the level of consciousness (LOC) of the subject is reduced to lower than 70% as compared to the LOC measured when the subject is awake such that a REM Sleep state is induced in the subject;
monitoring REM electroencephalogram (EEG) frequency patterns, electromyelogram (EMG) activity, and ocular muscle (OM) movement of the subject in the REM Sleep state such that a REM Profile of the subject is created;
determining a dream state or cognition activity of the subject while in the REM sleep state; and
correlating the REM Profile to the dream state and cognition activity of the subject to create a REM Index.

110. The method of claim 109, wherein monitoring further comprises recording parameters of the REM profile.

111. The method of claim 109, wherein monitoring further comprises recording patterns and frequencies of at least the REM EEG frequency patterns.

112. The method of claim 109, wherein determining comprises classifying the dream state as a positive dream, negative dream, or neutral dream.

113. The method of claim 109, wherein determining comprises classifying the cognition process as a learning process or memory formation.

114. The method of claim 109, further comprising measuring a REM Intensity of the subject, which comprises measuring REM sleep wave frequency in cycles per second.

115. The method of claim 109, further comprising indicating to a practitioner when a preset REM Profile correlated with a catalogued profile stored in a REM Index has been achieved by the subject.

116. The method of claim 112, further comprising correlating the EMG activity and OM movement of the subject to the dream state or cognition activity.

117. The method of claim 112, further comprising compiling a REM Index database of at least the REM EEG frequency patterns correlated to categorized dream states or cognition activity.

118. The method of claim 109, further comprising administering to the subject a pharmaceutically effective amount of a cognition or mood enhancing agent prior to, while, or after the REM Sleep state is maintained such that synaptic plasticity in neurons of the subject are created or enhanced.

119. The method of claim 118, further comprising compiling a REM Index database of at least the REM EEG frequency patterns correlated to cognition or mood enhancement activity.

120. A method for identifying a dream state and/or cognition activity of a subject undergoing REM Sleep, comprising:
monitoring REM electroencephalogram (EEG) frequency patterns, electromyelogram (EMG) activity, and ocular muscle (OM) movement of the subject induced in the REM Sleep state such that a REM profile of the subject is created;
comparing at least the REM EEG frequency patterns of the REM Profile to a database which correlates REM Profiles to categorized dream states or cognition activity while in the REM sleep state; and
identifying a dream state or cognition activity of the subject based upon the correlated REM Profile.

121. The method of claim 120, wherein monitoring further comprises recording cognition activity of the REM Profile.

122. The method of claim 120, wherein monitoring further comprises recording patterns and frequencies of at least the REM EEG frequency patterns of the subject to a REM Index.

123. The method of claim 120, wherein categorized dream states comprises positive dreams, negative dreams, and neutral dreams.

124. The method of claim 120, wherein categorized cognition activity comprises dream states indicative of memory and learning processes.

125. The method of claim 120, further comprising correlating the EMG activity and OM movement of the subject to the categorized dream states, cognition activity, and/or emotional state.

126. The method of claim 120, further comprising actuating an alarm when a predetermined dream state or cognition activity is achieved.

127. A method for inducing dreams in a subject, comprising:
administering to the subject a pharmaceutically effective amount of a first hypnotic agent such that the level of consciousness (LOC) of the subject is reduced to lower than 70% as compared to the LOC measured when the subject is awake such that a Continuous REM Sleep state is induced in the subject; and
measuring in the subject at least two of four variables including i) level of consciousness (LOC), ii) electromyelogram (EMG) activity, iii) ocular muscle (OM) movement, and iv) presence of specific REM electroencephalogram (EEG) waveforms indicative of a REM-like state; and
confirming a dream state in the subject based upon the at least two of four variables.

128. The method of claim 127, further comprising:
administering a second hypnotic agent to the subject continuously at a dose such that the subject remains in hypnosis and the LOC of the subject is maintained during this period of time at about 50-90%, 60-85%, 65-85%, or 70-80% as compared to the LOC measured when the subject is awake, thereby maintaining a Continuous REM Sleep state of the subject.

129. The method of claim 128, wherein the first and second hypnotic agents are the same.

130. The method of claim 128, wherein the first and second hypnotic agents are different.

131. The method of claim 128, wherein the first or the second hypnotic agent is a GABA-A agonist.

132. The method of claim 128, wherein the first or the second hypnotic agent is propofol, or its prodrug, metabolite, analog, or derivative.

133. The method of claim 128, wherein the second hypnotic agent is administered to the subject continuously at a dose such that the subject remains in hypnosis and the LOC of the subject is maintained at 70% to 80% with electromyelogram (EMG) activity present, ocular muscle movement and REM-characteristic electroencephalogram (EEG) waveforms.

134. The method of claim 128, wherein the first or second hypnotic agent is selected from the group consisting of benzodiazepines, cyclopyrrones, neurosteroids, barbiturates, etomidate, propofol, narcotics, fospropofol and ketamine.

135. The method of claim 128, further comprising: administering to the subject or person a neurological agent that is different from the first or second hypnotic agent.

136. The method of claim 135, wherein the neurological agent is selected from anti-emetics, local anesthetics, steroids, benzodiazepines, neurotransmitters, catecholoamines, serotonin uptake inhibitors, vasopressors, narcotics, anti-depressants and anti-psychotic medications, acetylcholinesterase inhibitors, nicotinic agonists, serotonin uptake inhibitors, glucocorticoids, GABA agonists, NMDA antagonists, NMDA agonists, antipsychotics, ampakines, calcium channel blockers, excitatory amines, monamine oxidase inhibitors, adenosine antagonists, phosphodiesterase inhibitors, noradrenaline uptake inhibitors, monamines, amphetamines, sympathomimetic amines, antidepressants, cerebral vasodilators, ergot derivatives, pyrrolidinones, free radical scavengers, and neuropeptides.

137. The method of claim 135, wherein the neurological agent is selected from the group consisting of ondansetron, dexamethasone, lidocaine, fentanyl, and midazolam.
